SEQUENCE LISTING (13 sequences)

SEQ ID NO: 1 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 2 - protein, 40 aa, Homo sapiens
MWVPVVFLTLSVTWIGAAPLILSRIVGGWECEKHSQPWQV

SEQ ID NO: 3 - protein, 40 aa, Homo sapiens
LVASRGRAVCGGVLVHPQWVLTAAHCIRNKSVILLGRHSL

SEQ ID NO: 4 - protein, 40 aa, Homo sapiens
FHPEDTGQVFQVSHSFPHPLYDMSLLKNRFLRPGDDSSHD

SEQ ID NO: 5 - protein, 40 aa, Homo sapiens
LMLLRLSEPAELTDAVKVMDLPTQEPALGTTCYASGWGSI

SEQ ID NO: 6 - protein, 40 aa, Homo sapiens
EPEEFLTPKKLQCVDLHVISNDVCAQVHPQKVTKFMLCAG

SEQ ID NO: 7 - protein, 40 aa, Homo sapiens
RWTGGKSTCSGDSGGPLVCNGVLQGITSWGSEPCALPERP

SEQ ID NO: 8 - protein, 21 aa, Homo sapiens
SLYTKVVHYRKWIKDTIVANP

SEQ ID NO: 9 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 10 - protein, 45 aa, Human papillomavirus type 16
MHQKRTAMFQDPQERPRKLPQLCTELQTTIHDIILECVYCKQQLL

SEQ ID NO: 11 - protein, 35 aa, Human papillomavirus type 16
RREVYDFAFRDLCIVYRDGNPYAVCDKCLKFYSKI

SEQ ID NO: 12 - protein, 35 aa, Human papillomavirus type 16
SEYRHYCYSLYGTTLEQQYNKPLCDLLIRCINCQK

SEQ ID NO: 13 - protein, 43 aa, Human papillomavirus type 16
PLCPEEKQRHLDKKQRFHNIRGRWTGRCMSCCRSSRTRRETQL

(19) United States
(12) Patent Application Publication
(10) Pub. No.: US 2018/0028627 A1
(43) Pub. Date: Feb. 1, 2018

(54) TREATMENT VACCINE FOR PROSTATE CANCER

(71) Applicant: Bioventures, LLC, Little Rock, AR (US)

(72) Inventors: Mayumi Nakagawa, Little Rock, AR (US); Al-Ola Abdallah, Overland Park, KS (US)

(21) Appl. No.: 15/552,793

(22) PCT Filed: Feb. 26, 2016
(86) PCT No.: PCT/US2016/019739

§ 371 Date: Aug. 22, 2017

Related U.S. Application Data
(60) Provisional application No. 62/120,991, filed on Feb. 26, 2015.

(51) Int. Cl. A61K 39/00; A61K 47/18; A61K 9/00

(52) U.S. Cl. CPC A61K 39/0011; A61K 9/0021; A61K 47/183; A61K 39/0002; A61K 2039/54; A61K 2039/58; A61K 2039/572

(57) ABSTRACT

A method of treating prostate cancer by intradermally injecting a person with a composition comprising (a) a recall antigen and (b) prostate specific antigen (PSA) or peptides that are fragments of PSA is provided. The recall antigen may be Candida extract, Trichophyton antigens, or mumps antigen, or other recall antigen.

### BACKGROUND

Prostate cancer is the most common cancer and the second leading cause of cancer-related death among the male population in Western countries.

Provenge is one currently available prostate cancer treatment. It is an autologough dendritic cell-based immunotherapy for late stage prostate cancer. This requires a laborious process of collecting peripheral blood mononuclear cells from each patient and generating autologous dendritic cells from them in a distant laboratory and loading the dendritic cells with a prostate cancer antigen before reinfusing them into the patient.

### SUMMARY

A novel therapeutic vaccine for prostate cancer is provided consisting of prostate specific antigen (PSA) peptides mixed with Candida skin test reagent. It is to be administered intradermally.

CANDIN is an extract of Candida albicans that is used as a skin test antigen to test immunocompetence of test subjects. Candida albicans is a yeast that is responsible for most oral thrush and vaginal yeast infections. Virtually every person has been exposed to Candida albicans, so it can be used to test for immune competency. CANDIN is intradermally injected in patients, and most persons with healthy immune systems will develop induration on the skin at the injection site indicative of a delayed-type-hypersensitivity response.

We have separately shown that CANDIN is a promising vaccine adjuvant for promoting T cell responses in a therapeutic vaccine for human papilloma virus (HPV). (U.S. Pat. No. 8,926,961; International Patent Application No. PCT/US14/60198, filed Oct. 11, 2014; and Wang, X., et al. 2013, Candida skin test reagent as a novel adjuvant for a human papillomavirus peptide-based therapeutic vaccine. Vaccine 31:5806-13). It induces interleukin 12 secretion in vitro by Langerhans cells, the main antigen-presenting cells in skin, and it has been found to promote T-helper type 1 (Th1) response in vaccine recipients.

PSA is widely used as a blood marker to screen for prostate cancer. PSA is expressed at lower levels in healthy men, but is usually expressed at higher levels in prostate cancer. It is not entirely specific to cancer, but is a good antigen for the vaccine because it is specific to prostate tissue and is elevated in prostate cancer.

Accordingly we have developed a vaccine comprising (1) Candida extract or other recall antigen, and (2) PSA (SEQ ID NO:1) or one or more antigenic fragments of PSA. The vaccine is intended for intradermal injection.

One embodiment of the vaccine comprises CANDIN and six PSA peptides consisting of residues 1-40, 41-80, 161-200, 81-120, 201-240, and 241-261 of SEQ ID NO:1. Each peptide will be acetylated at its amino terminus and amidated at its carboxy terminus. The unit dose composition will have 300 ul of CANDIN mixed with peptides, each in an amount of 50 to 500 ug in a total volume of up to 1 ml.

One embodiment of the invention provides a composition comprising: (a) a recall antigen, and (b) one or more purified polypeptides or peptides comprising antigens specific for prostate tissue or prostate cancer; and (c) a pharmaceutically acceptable diluent; wherein the composition is sterile.

One embodiment provides a method of making a composition comprising: (a) a recall antigen, and (b) one or more purified polypeptides or peptides comprising antigens specific for prostate tissue or prostate cancer; and (c) a pharmaceutically acceptable diluent; wherein the composition is sterile; the method comprising filling vials with the composition in a sterile clean room.

Another embodiment provides a method of treating prostate cancer comprising: intradermally injecting a person afflicted with prostate cancer with a composition comprising: (a) a recall antigen, and (b) one or more purified polypeptides or peptides comprising antigens specific for prostate tissue or prostate cancer; and (c) a pharmaceutically acceptable diluent; wherein the composition is sterile.

Another embodiment provides a composition comprising: (a) a plurality of antigenic peptide fragments of SEQ ID NO:1, wherein the peptide fragments are each 15-50 amino acid residues in length and collectively comprise residues 1-120 and 161-261 of SEQ ID NO:1: and (b) a pharmaceutically acceptable diluent; wherein the composition is sterile.

Another embodiment provides a method of preventing growth of prostate cancer tumors or preventing recurrence of prostate cancer in a mammal comprising: injecting intradermally into a mammal in need thereof a composition comprising: (a) a recall antigen, and (b) one or more purified polypeptides or peptides comprising antigens specific for prostate tissue or prostate cancer; and (c) a pharmaceutically acceptable diluent; wherein the composition is sterile; wherein the mammal is afflicted with prostate cancer, or the mammal was afflicted with prostate cancer and the cancer is now in remission.

### BRIEF DESCRIPTION OF THE DRAWINGS

FIGS. 1A-C Surface expressions of CD1a (FIG. 1A), Langerin (FIG. 1B), and E-cadherin (FIG. 1C) show successful conversion to LCs (solid lines). The dotted lines represent the relevant isotype controls.

FIGS. 2A-B Maturation effects on LCs examined by surface expression of CD40, CD80, CD86, and HLA-DR. (FIG. 2A) Representative FACS histograms from subject 2. The shaded gray area, the black dotted line, the black solid line, the short dashed line and the long dashed line represent the isotype control, media, CANDIN, “peptides” and CANDIN/“peptides” respectively. (FIG. 2B) Summary of results from all subjects examined.

FIG. 3 T-cell proliferation measured using alamarBlue. CANDIN and CANDIN/“peptides” pulsed LCs induce significantly increased T-cell proliferation compared to media. All wells contained CD3 T-cells (1.5×10⁵ cells) and autologous LCs (3×10³ cells).

FIG. 4 Representative results of cytokine expression by LCs treated with CANDIN (150 μl/ml) or CANDIN/“peptides” from subject 4 are shown. The bars represent SD of the replicates.

FIGS. 5A-I. Intracellular cytokine staining for IFN-γ, IL-4 and IL-17A of CD4 T-cells stimulated with LCs pulsed with CANDIN or CANDIN/“peptides”. (A) A representative dot plot for subject 1 showing the gating on lymphocytes. (B) A representative dot plot for subject 1 showing gating on live cells discriminated using eFluor 450. (C) A representative dot plot for subject 1 showing IL-4 secreting CD4 cells that were exposed to LCs pulsed with CANDIN/“peptides”. (D) Corresponding isotype control for IL-4. (E) A representative dot plot for subject 1 showing IFN-γ secreting CD4 cells that were exposed to LCs pulsed with CANDIN/“peptides”. (F) Corresponding isotype control for IFN-γ. (G) A representative dot plot showing IL-17A secreting CD4 cells that were exposed to LCs pulsed with CANDIN/“peptides”. (H) Corresponding isotype control for IL-17A. (I) Diagrams summarizing the results from all subjects.

FIG. 6A, circulating immune cells before, after 2, and after 4 vaccinations in all vaccine recipients. FIG. 6B, circulating immune cells in responders () and non-responders (▪). Percentages of CD4 cells positive for CD4 and Tbet were classified as Th1 cells, positive for CD4 and GATA3 were classified as Th2 cells, and positive for CD4, CD25, and FoxP3 were classified as Tregs. The bars represent standard error of means.

FIG. 7. Regulatory T-cells in lesional cervical epithelium and the underlying stroma. FoxP3 nuclear staining cells, in lesions (cervical intraepithelial neoplasia 1, 2, and/or 3) remaining after vaccination or representative region if no lesions remaining, were counted. The FoxP3 nuclear staining cells were also counted in the underlying stroma. The bars represent stand error of means.

FIG. 8. Fluorescent-activated cell sorting (FACS) detection of flow cytometry results of cell surface markers of Langerhans cell maturation: CD40, CD80, CD86, and HLA-DR. Monocytes were matured in maturation media with the indicated PSA peptides or peptide mixture, then subjected to FACS analysis.

FIG. 9. Enzyme-linked immunospot (ELISPOT) assay detecting interferon-gamma secretion from peripheral blood mononuclear cells (PBMCs) isolated from prostate cancer patients, and then stimulated with individual PSA peptides or the 6-peptide mixture.

### DETAILED DESCRIPTION

Example 1 below shows that in vitro CANDIN induces T-cell proliferation and interleukin-12 secretion by Langerhans cells (Example 1). This suggests CANDIN can promote activation of Langerhans cells, the most abundant antigen-presenting cells in the body, which are predominantly located in the skin.

Patients receiving a vaccine by intradermal injection comprising CANDIN mixed with peptides from a human papilloma virus protein, E6, had regression of precancerous HPV lesions and had an increase in Th1 response (Example 2).

Those findings lead us to believe that CANDIN mixed with antigenic peptides containing epitopes found in prostate cancer could induce an effective immune response to prostate cancer.

PROVENGE is an approved dendritic cell vaccine for prostate cancer, which shows that it is possible to get an immune response to prostate cancer tumors that is effective in causing tumor regression.

We produced 6 peptides that together cover 85% of the sequence of PSA. We showed in Example 4 that they can be solubilized together in one composition. Example 4 also shows that 4 of 10 naïve prostate cancer patients (not vaccinated with the present vaccine) have T cells that recognize and respond to (secrete interferon-gamma in response to) at least one of the 6 PSA peptides. This shows that even though PSA is a self protein, it is possible to induce a T cell immune response to PSA, and indeed that patients produce an immune response to PSA naturally, without any vaccination or treatment. This, together with the results found with the CANDIN-HPV peptide vaccine mixture, that intradermal vaccination with a mixture of CANDIN and PSA peptides or PSA protein could boost a T cell response to PSA, and that might be effective to treat prostate cancer.

One embodiment provides a composition comprising: (a) a recall antigen, and (b) one or more purified polypeptides or peptides comprising antigens specific for prostate tissue or prostate cancer; and (c) a pharmaceutically acceptable diluent, wherein the composition is sterile.

The prototypical recall antigens are those commonly used in immunologic skin testing to test immune response, particularly mumps antigen, candida antigen, and trichophyton antigen. The test shows if the body “remembers” or “recalls” the antigen, i.e., has a delayed-type hypersensitivity response in the skin where the antigen was administered by intradermal injection.

The term “recall antigen” is defined herein as a substance or mixture containing a plurality of proteinaceous antigens, wherein the mixture induces a delayed-type hypersensitivity response in intradermal skin test in a majority of people previously sensitized or exposed to the recall antigen. The prototypical recall antigens are those commonly used in immunologic skin testing to test immune response, particularly mumps antigen, candida antigen, and trichophyton antigen. Each of these, although referred to by the singular term “antigen” is actually composed of several or many molecular substances that can induce an immune response.

In specific embodiments, the recall antigen may be mumps antigen (e.g., killed whole mumps virus), Candida extract, or Trichophyton extract.

In specific embodiments, the recall antigen is killed whole virus, killed whole bacteria, or killed whole microorganisms. In specific embodiments, it is an extract of fungi. The term “fungi” here includes yeasts.

CANDIN is a preferred recall antigen. Other recall antigens can be substituted for CANDIN. These include, for example mumps antigen and trichophyton extract. The amount and concentration of the recall antigen should be at least enough to induce dermal induration upon intradermal injection in a majority of immunocompetent adult humans.

Component (b) of the composition is one or more purified polypeptides or peptides comprising antigens relatively specific for prostate tissue or prostate cancer. A preferred polypeptide is PSA (SEQ ID NO:1).

The component (b) may be the whole polypeptide (for instance the whole PSA polypeptide). In other embodiments, it may be one or more peptides of 10-100 residues of PSA or of another polypeptide that is specific for prostate tissue or prostate cancer.

In other specific embodiments, instead of PSA peptides, the entire PSA protein (SEQ ID NO:1) may be used in the composition.

```
Prostate specific antigen (PSA) (SEQ ID NO: 1):
                10 20 30 40 50
MWVPVVFLTL SVTWIGAAPL ILSRIVGGWE CEKHSQPWQV LVASRGRAVC
                60 70 80 90 100
GGVLVHPQWV LTAAHCIRNK SVILLGRHSL FHPEDTGQVF QVSHSFPHPL
              110 120 130 140 150
YDMSLLKNRF LRPGDDSSHD LMLLRLSEPA ELTDAVKVMD LPTQEPALGT
              160 170 180 190 200
TCYASGWGSI EPEEFLTPKK LQCVDLHVIS NDVCAQVHPQ KVTKFMLCAG
              210 220 230 240 250
RWTGGKSTCS GDSGGPLVCN GVLQGITSWG SEPCALPERP SLYTKVVHYR
              260
KWIKDTIVAN P
```

### Candin

CANDIN® (candida albicans) is made from the culture filtrate and cells of two strains of Candida albicans. The fungi are propagated in a chemically defined medium consisting of inorganic sals, biotin and sucrose. Lyophilized source material is extracted with a solution of 0.25% NaCl, 0.125% NaHCO₃ and 50% v/v glycerol. The concentrated extract is diluted with a solution of 0.5% NaCl, 0.25% NaHCO₃, 0.03% Albumin (Human) usp, 8 ppm polysorbate 80 and 0.4% phenol.

The potency of CANDIN® (Candida albicans extract) is measured by DTH skin tests in humans. The procedure involves concurrent (side-by-side) testing of production lots with an Internal Reference (IR), using sensitive adults who have been previously screened and qualified to serve as test subjects. The induration response at 48 hours elicited by 0.1 mL of a production lot is measured and compared to the response elicited by 0.1 mL of the IR. The test is satisfactory if the potency of the production lot does not differ more than ±20% from the potency of the IR, when analyzed by the paired t-test (two-tailed) at a p value of 0.05

The potency of the IR is monitored by DTH skin testing. Persons included in the potency assay are qualified as test subjects by receiving four skin tests with the IR from which a mean induration response (mm) is calculated. Current skin tests with the IR must show that the potency of the IR has not changed more than ±20% from the mean qualifying response in the same test subjects, when analyzed by the paired t-test (two-tailed) at a p value of 0.05. The required induration response at 48 hours to the IR is 15 mm ±20%.

The skin-test strength of CANDIN® (candida albicans) has been determined from dose-response studies in healthy adults. The product is intended to elicit an induration response ≧5 mm in immunologically competent persons with cellular hypersensitivity to the antigen.

### Embodiments

One embodiment provides a composition comprising: (a) a recall antigen, and (b) one or more purified polypeptides or peptides comprising antigens specific for prostate tissue or prostate cancer; and (c) a pharmaceutically acceptable diluent; wherein the composition is sterile.

In a specific embodiment, component (b) is a purified polypeptide comprising prostate specific antigen (SEQ ID NO:1) or is one or more purified antigenic peptide fragments of SEQ ID NO:1.

In a more specific embodiment, the component (b) is one or more purified antigenic peptide fragments of SEQ ID NO:1, wherein the peptide fragments are 10-80 amino acid residues in length. In other embodiments, they are 10-50, or 20-50 amino acid residues in length.

In a specific embodiment, the one or more purified antigenic peptide fragments are selected from peptides consisting of residues 1-40, residues 41-80, 81-120, residues 161-200, residues 201-240, and residues 241-261 of SEQ ID NO:1.

In a specific embodiment, component (b) is a plurality of antigenic peptide fragments of SEQ ID NO:1, wherein the peptide fragments are each 15-50 amino acid residues in length and collectively comprise residues 1-120 and 161-261 of SEQ ID NO:1.

In a more specific embodiment, the plurality of antigenic peptide fragments of SEQ ID NO:1 do not collectively comprise residues 121-160 of SEQ ID NO:1. This means that they do not collectively comprise the entirety of residues 121-160. They may comprise a portion of this sequence. In other embodiments, they do not comprise any portion of the sequence of residues 121-160 of SEQ ID NO:1.

In a specific embodiment of the composition, component (b) consists of peptide fragments of SEQ ID NO:1 consisting of residues 1-40, 41-80, 81-120, 201-240, and 241-261 of SEQ ID NO:1.

In specific embodiments of the composition, the peptides are acetylated on their N-terminus or amidated on their C-terminus, or both acetylated on their N-terminus and amidated on their C-terminus.

In specific embodiments, the recall antigen is candida extract, mumps antigen, or trichophyton extract.

In specific embodiments where component (b) consists of peptide fragments of SEQ ID NO:1, the peptide fragments collectively comprise more than 50% of the sequence of SEQ ID NO:1.

In specific embodiments of the compositions, the composition is an aqueous solution or suspension having a pH below 6.0. In more specific embodiments, the composition has a pH of between 3.0 and 6.0.

In specific embodiments, the composition comprises 1% to 12% (weight/volume) glycine. In other specific embodiments of the compositions, the composition comprise 2% to 12%, 3% to 10%, or 3% to 7% glycine.

Another embodiment provides a method of making a composition of the invention comprising filling vials with the composition in a sterile clean room.

Another type of composition is a composition lacking the recall antigen. The PSA polypeptide or peptide mixture can be prepared separately from the recall antigen and then mixed with the recall antigen. For instance, a mixture of PSA peptides can be prepared as a solution in 10 mM succinate, 5% glycine, pH 5.0, in a sterile solution with a pharmaceutically acceptable diluent. And then the PSA peptide solution can be mixed with CANDIN immediately before intradermally injecting the mixture.

Thus, one embodiment provides a composition comprising: (a) a plurality of antigenic peptide fragments of SEQ ID NO:1, wherein the peptide fragments are each 15-50 amino acid residues in length and collectively comprise residues 1-120 and 161-261 of SEQ ID NO:1; and (b) a pharmaceutically acceptable diluent; wherein the composition is sterile.

In specific embodiments, the plurality of antigenic peptide fragments of SEQ ID NO:1 do not collectively comprise residues 121-160 of SEQ ID NO:1.

In specific embodiments, the composition is an aqueous solution or suspension having a pH below 6.0, or has a pH of between 3.0 and 6.0.

In specific embodiments, the composition comprises 2% to 12% (w/v) glycine.

In specific embodiments, the composition further comprises a recall antigen.

Another embodiment provides a method of making a composition of the invention comprising filling vials with the composition in a sterile clean room.

Another embodiment provides a method of treating prostate cancer comprising: intradermally injecting a person afflicted with prostate cancer with a composition comprising: (a) a recall antigen, and (b) one or more purified polypeptides or peptides comprising antigens specific for prostate tissue or prostate cancer; and (c) a pharmaceutically acceptable diluent; wherein the composition is sterile.

In a specific embodiment of that method, the prostate cancer in the person is in partial or complete remission at the time of the intradermally injecting step, and the method is a method of preventing growth of the cancer or preventing recurrence of the cancer.

Another embodiment provides a method of preventing growth of prostate cancer tumors or preventing recurrence of prostate cancer in a mammal comprising: injecting intradermally into a mammal in need thereof a composition comprising: (a) a recall antigen, and (b) one or more purified polypeptides or peptides comprising antigens specific for prostate tissue or prostate cancer; and (c) a pharmaceutically acceptable diluent; wherein the composition is sterile; wherein the mammal is afflicted with prostate cancer, or the mammal was afflicted with prostate cancer and the cancer is now in remission.

Another embodiment provides a composition comprising: (a) a plurality of antigenic peptide fragments of SEQ ID NO:1, wherein the peptide fragments are each 15-50 amino acid residues in length and collectively comprise residues 1-120 and 161-261 of SEQ ID NO:1: and (b) a pharmaceutically acceptable diluent; wherein the composition is sterile.

### EXAMPLES

### Example 1

### Solubilizing Amidated and Acetylated HPV E6 81-115 Peptide, and Formation of Pharmaceutical Formulation

We attempted to make a pharmaceutical formulation with four peptides from human papilloma virus (HPV) strain 16 protein E6. The 4 peptides were peptides consisting of residues 1-45, 46-80, 81-115, and 116-158 of SEQ ID NO:9, which is the HPV 16 E6 protein. Each of the peptides was amidated at its carboxyl terminus and acetylated at its amino terminus. The peptides were each chemically synthesized.

The HPV 16 E6 81-115 peptide was found to be insoluble in any suitable buffer for manufacturing. However, it was found that it could be solubilized and will stay soluble when added to 10 mM glutamate, pH 4.0 solution which already contains solubilized E6 1-45, E6 46-80, and E6 116-158 at 5 mg/ml concentration for each of the four peptides.

For the pharmaceutical formulation, this was mixed with trehalose as a stabilizing agent and glycine as tonicity modifier. The final concentrations of the formulation were 10 mM glutamate, 1.0% w/v trehalose, 2.0% w/v glycine, and 0.714 mg/ml each of the four peptides.

The formulation was lyophilized for storage, and reconstituted immediately before use by addition of the appropriate volume of water for injection to produce the concentrations stated above.

### Example 2

### Candida Skin Test Reagent as a Novel Adjuvant for a Human Papilloma Virus Peptide-Based Therapeutic Vaccine

A vaccine adjuvant that can effectively promote cell-mediated immunity is currently not available. Because of the ability of a Candida skin test reagent injection to induce common wart regression, our group is using it as a novel adjuvant in a clinical trial of a peptide-based human papillomavirus therapeutic vaccine. The goal of this current study was to investigate the mechanisms of how Candida enhances the vaccine immune responses. Maturation effects on Langerhans cells, capacity to proliferate T-cells, expression of cytokines and pattern recognition receptors by Langerhans cells, and ability to induce Th1, Th2, and Th17 responses were investigated in healthy subjects. The vaccine, human papillomavirus peptides with Candida, demonstrated partial maturation effects on Langerhans cells indicated by significantly up-regulated CD40 (p=0.00007) and CD80 (p<0.00001) levels, and showed T-cell proliferative capacity (p<0.00001) when presented by Langerhans cells in vitro. Interestingly, the maturation effects were due to the peptides while Candida was responsible for the T-cell proliferation. The cytokine profile (IL-1β, IL-6, IL-8, IL-10, IL-12p40, IL-23Ap19 , IFN-γ, and TNF-α) of Langerhans cells treated with the vaccine or Candida alone showed that IL-12p40 mRNA was most frequently induced, and IL-12p70 protein was detected in the supernatants. The presence of pattern recognition receptors known to associate with Candida albicans (DC-SIGN, dectin-1, dectin-2, galectin-3, mincle, mannose receptor, Toll-like receptors-1, 2, 4, 6, and 9) were demonstrated in all subjects. On the other hand, the induction of Th1 response demonstrated by IFN-γ secretion by CD4 cells stimulated with the vaccine or Candida pulsed Langerhans cells was demonstrated only in one subject. In summary, the Langerhans cell maturation effects of the vaccine were due to the peptides while the T-cell proliferative capacity was derived from Candida, and the most frequently induced cytokine was IL-12.

### Abbreviations

APCs, antigen presenting cells; HPV, human papillomavirus; LCs, Langerhans cells; MFI, mean fluorescence intensity; PAMPs, pathogen-associated molecular patterns; PBMC, peripheral blood mononuclear cells; PE, phycoerythrin; qRT-PCR, quantitative real-time PCR; PRRs, pattern recognition receptors.

### 1. Introduction

The most widely used adjuvant in approved human vaccines is an alum-based adjuvant that has been shown to elicit a predominantly Th2 immune response [1]. Therefore, the alum-based adjuvant would be useful in a vaccine designed to boost antibody responses, but not for a vaccine designed to stimulate cellular immune responses. Since successful clearance of human papillomavirus (HPV) infection is believed to be induced by cell-mediated immunity [2, 3], an adjuvant that would promote such an immunity is necessary, but not available.

Our group and others have shown that serial intra-lesional injections of common warts with skin testing reagents such as Candida, mumps, and/or Trichophyton can induce regression not only of treated warts but also of distant untreated warts [4-9]. In a Phase I clinical trial (NCT00569231), our group used intralesional injection of CANDIN (Allermed, San Diego, Calif.), a colorless extract of Candida albicans, to treat common warts. Resolution of treated warts occurred in 82% of the subjects, and anti-HPV T-cell responses were demonstrated [8]. Given that CANDIN is derived from C. albicans, it should contain numerous pathogen-associated molecular patterns (PAMPs). We hypothesized that CANDIN would be an effective vaccine adjuvant which would stimulate multiple pattern recognition receptors (PRRs) and induce innate as well as adaptive immunity.

Cervical cancer is almost always caused by high-risk HPV infection, and is the 2^{nd} most common cancer among women in the world. Two very effective prophylactic HPV vaccines, Gardasil® (Merck, N.J., USA) and Cervarix® (GlaxoSmithKline, Middlesex, UK), are available, and they work by inducing high titers of neutralizing antibody [10-12]. However, they are not effective for women with pre-existing HPV infection [10, 12, 13]. Therefore, a therapeutic HPV vaccine that can be used for those already infected with HPV and/or have developed HPV-associated neoplasia is not available. Our group studied naturally induced immunity in women with HPV infection and/or cervical lesions, and have found that the ability to induce T-cell responses against E6, one of the oncoproteins of high-risk HPVs, is associated with HPV clearance and regression of cervical lesions [3, 14, 15]. Therefore, we designed an HPV therapeutic vaccine which consists of four HPV type 16 E6 peptides and CANDIN, and are conducting a Phase I clinical trial (NCT01653249).

In the current study, we examined the immune enhancing effects of CANDIN as a vaccine adjuvant. Surprisingly, the E6 peptides were responsible for the partial maturation of Langerhans cells (LCs) while CANDIN was responsible for the T-cell proliferative effects. The most commonly induced cytokine by the LCs was IL-12.

### 2. Materials and Methods

### 2.0 Preparation of Primers

A mixture of the HPV 16 E6 peptides was prepared and solubilized as described in Example 1.

### 2.1 Generation of Monocytes-Derived LCs

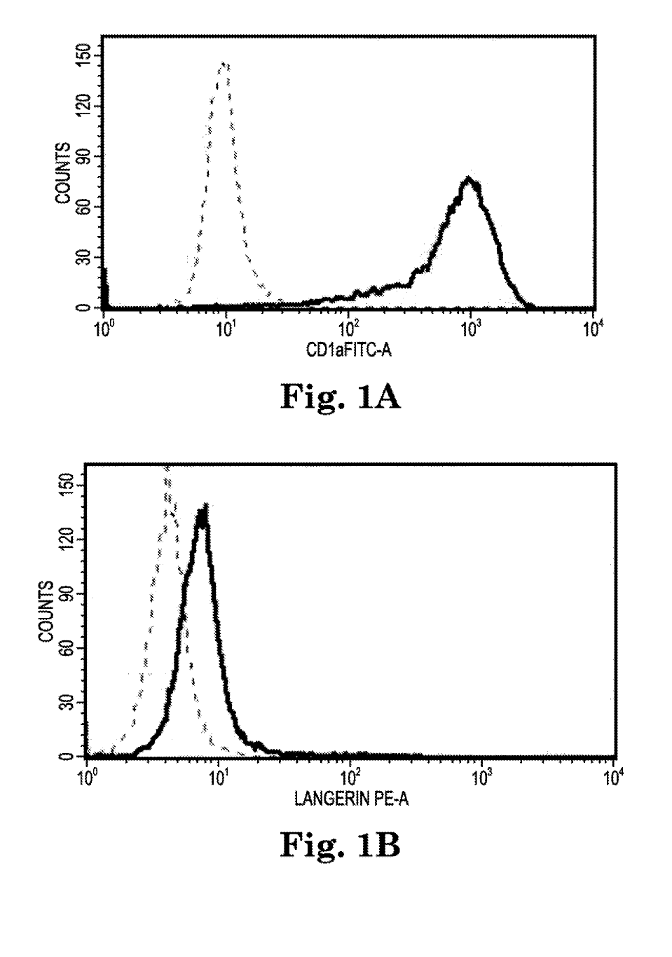

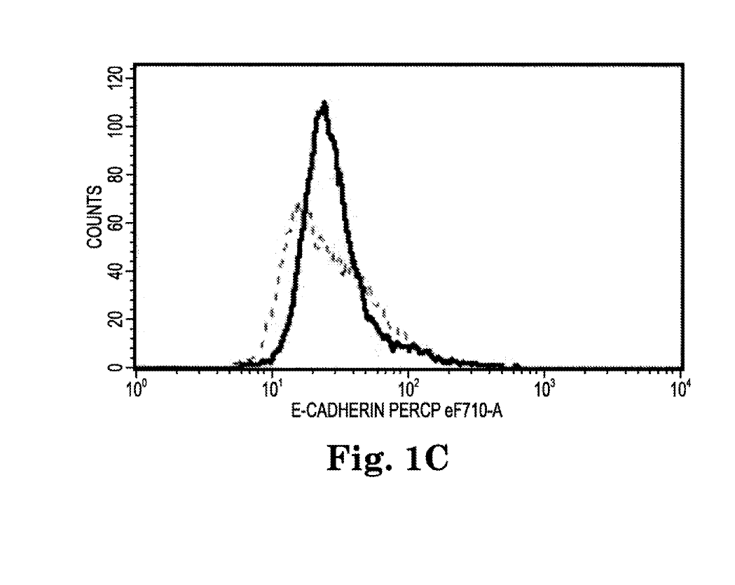

Mononuclear cells were collected from healthy blood donors (n=10) by apheresis (Key Biologics, LLC, Memphis, Tenn.). The subjects were numbered in a chronological order. Peripheral blood mononuclear cells (PBMCs) were purified using the ficoll gradient centrifugation method. Monocytes were negatively isolated from PBMC using Monocyte Isolation Kit II (Miltenyi Biotec, Auburn, Calif.), and were converted to LCs using granulocyte-macrophage colony-stimulating factor, IL-4, and transforming growth factor β-1 as described by Fahey et al. [17]. The effectiveness of conversion to LCs was demonstrated by detecting CD1a (eBioscience, San Diego, Calif.), Langerin (Beckman-Coulter, Brea, Calif.), and E-cadherin (eBioscience) using FACS Fortessa (University of Arkansas for Medical Sciences Microbiology and Immunology Flow Cytometry Core Laboratory) and CellQuest Pro software (BD Biosciences, San Jose, Calif.) in selected experiments (FIG. 1). Sufficient number of cells were available from all subjects except for subject 1 in whom the LC maturation experiment could not be performed.

### 2.2 Maturation Analysis of LCs Treated with CANDIN and/or HPV Peptides

CANDIN was dialyzed before use to remove a small amount of solvent (0.4% phenol) using Slide-A-Lyzer G2 Dialysis Cassette (Thermo Scientific, Rockford, Ill.). LCs were prepared as described above, and one million LCs each were treated with CANDIN (150 μl/ml), four current good manufacturing practice-grade HPV16 E6 peptides [E6 1-45, E6 46-80, E6 81-115, and E6 116-158 (referred to as “peptides” hereafter); 10 μg/ml/peptide; made by CPC Scientific, Sunnyvale, Calif. and vialed by Integrity Bio, Camarillo, Calif.], or CANDIN/“peptides”. Zymosan (10 μg/ml, InvivoGen, San Diego, Calif.), a yeast cell wall particle containing many polysaccharides including β-glucan and mannan [18], was used as a positive control. After 48 hour incubation, cells were stained with anti-human CD40 phycoerythrin (PE)-Cy5.5, CD80 fluorescein isothiocyanate, CD86 PE-Cy5 and HLA-DR PE (eBioscience, San Diego, Calif.). Ten thousand events were acquired, and the data were analyzed using Flowjo software (BD Biosciences).

### 2.3 Analysis of T Cell Proliferation Induced by LCs Treated with CANDIN and/or “Peptides”

On day 7 of LCs conversion, CD3 T cells from the same subjects were negatively isolated from PBMCs using Pan T-Cell Isolation Kit II (Miltenyi Biotec). To remove CD25 regulatory T cells, human CD25 Antibody-Biotin (Miltenyi Biotec) was added. T cell proliferation assay was performed in 6 replicate wells by co-culturing T cells (1.5×10⁶ cells/ml) with autologous LCs (3×10⁴ cells/ml) in 100 μl of complete Yssel's media (Gemini Bioproducts Inc, Woodland, Calif.) containing 1% human serum in each well of a 96-well plate. Wells containing cells only (T-cells and LCs), cells and CANDIN (150 μl/ml), cells and CANDIN/“peptides”, and cells and tetanus toxoid (500 ng/ml, EMD Milipore, Billerica, Mass.) were set up. After 7 days of incubation, 10 μl of alamarBlue (Life Technologies, Grand Island, N.Y.) was used to replace the corresponding volume of media in each well, then the plate was incubated at 37° C. for 6 hours. Fluorescence was measured (530 nm excitation wavelength and 590 nm emission wavelength) in media using BioTek Synergy-2 Multi Plate Reader (US BioTek, Seattle, Wash.).

### 2.4 Cytokine and PRR Analyses by Quantitative Real-Time PCR (qRT-PCR)

One million LCs each were treated with CANDIN (50 μl/ml, 100 μl/ml, and 150 μl/ml) with or without “peptides” (10 μg/ml/peptide) at each CANDIN concentration. Zymosan was used as a positive control at 10 ug/ml and media only as a negative control. Cells were harvested for RNA after 8 and 24 hours. RNA was extracted using RNeasy kit (Qiagen, Valencia, Calif.), and treated with DNase I (Promega, Madison, Wis.). cDNA synthesis was carried out using SuperScript III first-strand synthesis system (Life Technologies).

Quantitative PCR analysis was performed in duplicate for cytokines including IL10, IL6, IL8, IL10, IL-12p40, IL-23Ap19, IFN-γ and TNF-α using an iQ-SYBR mix (Bio-Rad, Hercules, Calif.). In addition, expressions of PRRs (DC-SIGN, dectin-1, dectin-2, galectin-3, mincle, mannose receptor, TLR-1, TLR-2, TLR-4, TLR-6, and TLR-9) known to associate with C. albicans [19-28] were examined. The primers used to detect IL-12 were previously reported by Vernal et al. [29]. All other primers were designed using Beacon Design software (Bio-Rad, Table 1). The threshold cycles were normalized to a human housekeeping gene, glyceraldehyde 3-phosphate dehydrogenase, and were calculated as fold change over untreated LCs at 8 hours. mRNA was considered to be detected when amplification of cDNA was demonstrated.

### 2.5 IL-12p70 Protein Analysis by ELISA

Supernants from LCs treated with CANDIN (50 μl/ml, 100 μl/ml and 150 μl/ml) with or without “peptides” (10 μg/ml/peptide) from the qRT-PCR experiments at 24 hours were collected and tested using the IL-12p70 High Sensitivity ELISA kit (eBioscience). Values from media only wells were subtracted from experimental wells.

### 2.6 Intracellular Cytokine Staining

The methods were adapted according to those described by Zielinski et al. [30]. CD4 T-cells were negatively isolated from PBMCs using CD4 T Cell Isolation Kit II (Miltenyi Biotec) and were co-cultured with autologous LCs at a ratio of 50:1 (CD4 T-cells:LCs). CANDIN (150 μl/ml) with or without “peptides” (10 μg/ml/peptide) were added to stimulate cells. Media alone was used as a negative control. After 6 days of co-culture, the cells were stimulated with phorbol 12-myristate 13-acetate (200 nM, Sigma, St. Louis, Mo.), and ionomycin (1 μg/ml, Sigma) for 2 hours. Then, Brefeldin A (10 μg/ml, eBioscience) was added for additional 2 hours. After being stained using fixable viability dye eFluor 450® (eBioscience), the cells were permeabilized/fixed and stained with anti-human IFN-γ PE, IL-17A peridinin chlorophyll protein-Cy5.5, IL-4 allophycocyanin, or relevant isotype controls (eBioscience). Ten thousand events were acquired using FACS Fortessa. Live lymphocytes were gated, and the percentages of IFN-γ, IL-17A and IL-4 positive CD4 T-cells were analyzed using FACS Diva (BD Biosciences) and Flowjo softwares.

### 2.7 Statistical Analysis

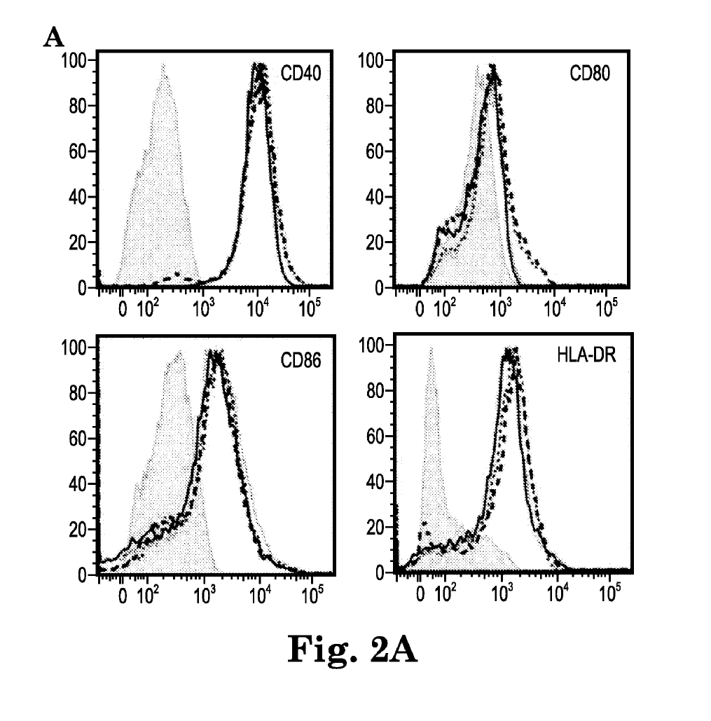

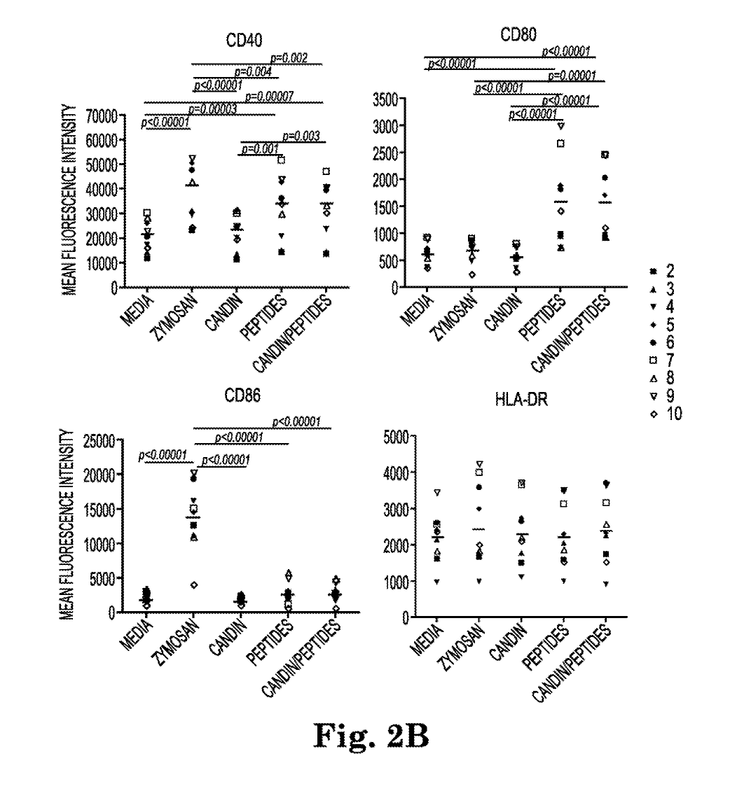

A mixed effects ANOVA was used to compare the groups while accounting for the dependence between groups. Tukey's multiple comparison procedure was used to perform all pairwise comparisons for maturation markers (FIG. 2B) while Dunnet's test was used to compare the media control values to the remaining groups for T-cell proliferation (FIG. 3).

### 3. Results

### 3.1 Phenotypic Maturation of LCs

We evaluated the maturation effects of CANDIN, and/or the E6 “peptides” on LCs (FIGS. 1-2). For CD40, statistically significant increases in mean fluorescence intensity (MFI) were observed with LCs treated with zymosan (p<0.00001), “peptides” (p=0.00003) and CANDIN/“peptides” (p=0.00007) compared to untreated LCs. In addition, MFIs of LCs treated with “peptides” and CANDIN/“peptides” were significantly higher than the MFI of LCs treated with CANDIN alone (p=0.001 and 0.003 respectively). For CD80, significant increases in MFIs were observed with LCs treated with “peptides” (p<0.00001) and CANDIN/“peptides” (p<0.00001) over media. Compared to CANDIN treated LCs, CD80 expression was significantly higher in “peptide” and CANDIN/“peptide” treated LCs (p<0.00001 for both). Only zymosan increased the MFI for CD86 significantly (p<0.00001). No significant increases were observed for HLA-DR. In summary, the “peptides” exerted partial LC maturation effects while CANDIN did not. Endotoxin levels for the “peptides” tested individually were all undetectable (<1.0 EU/mg).

### 3.2 T-cell Proliferation Measured with AlamarBlue

Proliferation was significantly increased with CANDIN (p<0.00001) and CANDIN/“peptides” (p<0.00001) over media (FIG. 3). “Peptides” did not induce measureable proliferation. Measurable proliferation with tetanus toxoid (increased fluorescence of ≧5000) was demonstrated in subjects 2 and 5, but overall no significant increase over media was observed (FIG. 3). Though unlikely, a possibility that LCs may have proliferated in addition to T-cells cannot be ruled out.

3.3 Expression of Cytokines by LCs Pulsed with CANDIN or CANDIN/“peptides”

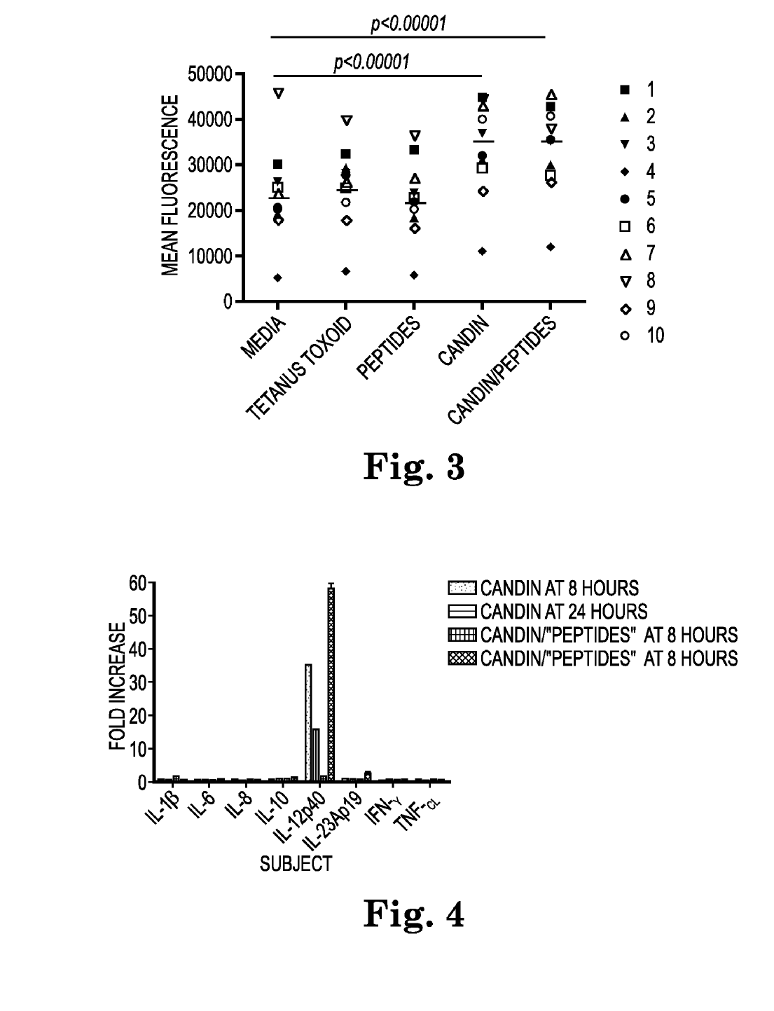

LCs from ten subjects were treated with CANDIN or CANDIN/“peptides”, and mRNA expression of 8 cytokines (Table 1) were examined by qRT-PCR (FIG. 4, Table 2). The amplifications of the intended products were confirmed by DNA sequencing after gel-purification from selected experiments. Overall, the cytokine expression profiles of LCs treated with CANDIN and CANDIN/“peptide” were similar. IL-12p40 was the most commonly enhanced cytokine (≧5 fold over untreated), and expression was detected in 5 subjects with CANDIN and in 7 subjects with CANDIN/“peptides”. IFN-γ was the 2^{nd} most commonly induced cytokine (6 subjects), and was detected in 5 subjects with CANDIN and in 4 subjects with CANDIN/“peptides”. IL10 was also induced in 6 subjects: 4 subjects with CANDIN and 6 subjects with CANDIN/“peptide”. IL6 and IL-23p19 were induced only with CANDIN (2 subjects for IL6 and 1 subject for IL-23p19.) TNF-α was expressed only with CANDIN/“peptide” in 1 subject. IL8 and IL10 were not expressed in any subjects.

Supernatants from LCs treated with CANDIN or CANDIN/“peptides” for 24 hours were analyzed for the presence of IL12p70 protein. IL12p70 was detected in 27 of 30 samples treated with CANDIN (range 38 to 177 ng/ml) and in 27 of 30 samples treated with CANDIN/“peptides” (range 38 to 299 ng/ml).

TABLE 1
Primers used for qRT-PCR

| | Gene | | Forward primer | Reverse primer |
|---|---|---|---|---|
| Description | name | Accession no. | sequence | sequence |
| Interleukin 1 beta | hIL-1β | NM_000576.2 | CAG GGA CAG | CAC GCA GGA |
| | | | GAT ATG GAG | CAG GTA CAG |
| | | | CAA C | ATT C |
| Interleukin 6 | hIL-6 | NM_000600.3 | GTA GTG AGG | GGC ATT TGT |
| (interferon, beta 2) | | | AAC AAG CCA | GGT TGG GTC |
| | | | GAG C | AGG |
| Interleukin 8 | hIL-8 | NM_000584.3 | GAC CAC ACT | AAA CTT CTC |
| | | | GCG CCA ACA C | CAC AAC CCT |
| | | | | CTG C |
| Interleukin 10 | hIL-10 | NM_000572.2 | GGG TTG CCA | CGC CGT AGC |
| | | | AGC CTT GTC TG | CTC AGC CTG |
| Interleukin 12B | hIL- | NM_002187.2 | CCC TGA CAT | AGG TCT TGT |
| | 12p40 | | TCT GCG TTC A | CCG TGA AGA |
| | | | | CTC TA |
| Interleukin 23 alpha | hIL23A | NM_016584.2 | AGT GTG GAG | GGG CTA TCA |
| subunit p19 (IL23A) | p19 | | ATG GCT GTG ACC | GGG AGC AGA |
| | | | | GAA G |
| interferon, gamma | hIFN-γ | NM_000619.2 | TGT GGA GAC | TGC TTT GCG |
| | | | CAT CAA GGA | TTG GAC ATT |
| | | | AGA C | CAA G |
| Tumor Necrosis | hTNF-α | NM_000594.3 | GGG GTG GAG | ACG GCG ATG |
| Factor alpha | | | CTG AGA GAT | CGG CTG ATG |
| | | | AAC C | |
| DC-SIGN, CD 209 | hDCSIGN | NM_001144899.1 | TGC AGT CTT CCA | TGT TGG GCT |
| | | | GAA GTA ACC | CTC CTC TGT |
| | | | GCT | TCC AAT |
| C-type lectin domain | hDectin1 | NM_197947.2 | TGC TTG GTA ATA | GGT TGA CTG |
| family 7, member A | | | CTG GTG ATA G | TGG TTC TCT T |
| (CLEC7A) | | | | |
| C-type lectin domain | hDectin2 | NM_001007033 | AAC ACA GAA | TCC AGA AGA |
| family 6, member A | | | GCA GAG CAG | CTA TTG AAG |
| (CLEC6A) | | | AAT | CAC ATT |
| Lectin, galactoside- | hGalectin3 | NM_001177388.1 | TGT GCC TTA TAA | TTC TGT TTG |
| binding, soluble, 3 | | | CCT GCC TTT GCC | CAT TGG GCT |
| (LGAL3) | | | | TCA CCG |
| C-type lectin domain | hMincle | NM_014358.2 | TCA GAA TAC | TGG TTA CAG |
| family 4, member E | | | CGG TGT GGC CTT | CCT GTT TGG |
| (CLEC4E) | | | TCT | AGC TGA |
| Mannose receptor, C | hMRC2 | NM_006039.4 | AGC AAC GTC | AGA ACT GTG |
| type2 | | | ACC AAA GAA | CCT CTG ACC |
| | | | ACG CAG | ACT TCA |
| Toll-Like Receptor | hTLR1 or | NM_003263.3 or | ATG TGG CAG CTT | TCT GGA AGA |
| 1/6* | TLR6 | NM_006068.4 | TAG CAG CCT TTC | AAT CAG CCG |
| | | | | ATG GGT |
| Toll-Like Receptor 2 | hTLR2 | NM_003264 | TGC TGC CAT TCT | CAC TCC AGG |
| | | | CAT TCT | TAG GTC TTG |
| Toll-Like Receptor 4 | hTLR4 | NM_138557 | CGT GCT GGT ATC | GGT AAG TGT |
| | | | ATC TTC AT | TCC TGC TGA G |
| Toll-Like Receptor 9 | hTLR9 | NM_017442.3 | ATC TGC ACT TCT | AAG GCC AGG |
| | | | TCC AAG GCC | TAA TTG TCA |
| | | | TGA | CGG AGA |
| Glyceraldehyde-3- | hGAPDH | NM_002046.4 | GGA CCT GAC | GTA GCC CAG |
| phosphate | | | CTG CCG TCT AG | GAT GCC CTT GA |
| dehydrogenase | | | | |

*The same primers were used to analyze TLR 1 and 6 amplifying a 100% homologous region between the two genes.

### 3.4 Expression of PRRs on LCs

All 11 PRRs examined were detectable in untreated LCs of all subjects (data not shown). Upon stimulation with CANDIN or CANDIN/“peptides”, few PRRs showed increased expression (≧5 fold over untreated). No obvious differences were observed in PRRs expressed between CANDIN- and CANDIN/“peptide”-treated LCs. The expression of TLR-9 was increased in 3 subjects (5 to 18 fold with CANDIN and 9 to 16 fold with CANDIN/“peptides”), mincle in 2 subjects (5 fold with CANDIN and CANDIN/“peptides”), mannose receptor in 2 subjects (5 to 9 fold with CANDIN and 5 to 11 fold with CANDIN/“peptides”), dectin-2 in 2 subjects (5 to 54 fold with CANDIN and 5 to 8 fold with CANDIN/“peptides”), and DC-SIGN in 1 subject (5 to 22 fold with CANDIN). In 5 subjects with increased expression of PRRs, 3 of them showed the increased expressions of two or more PRRs in LCs.

### 3.5 Intracellular Cytokine Expression of CD4 T-cells Stimulated with CANDIN-Pulsed LCs or CANDIN/“Peptides”-Pulsed LCs

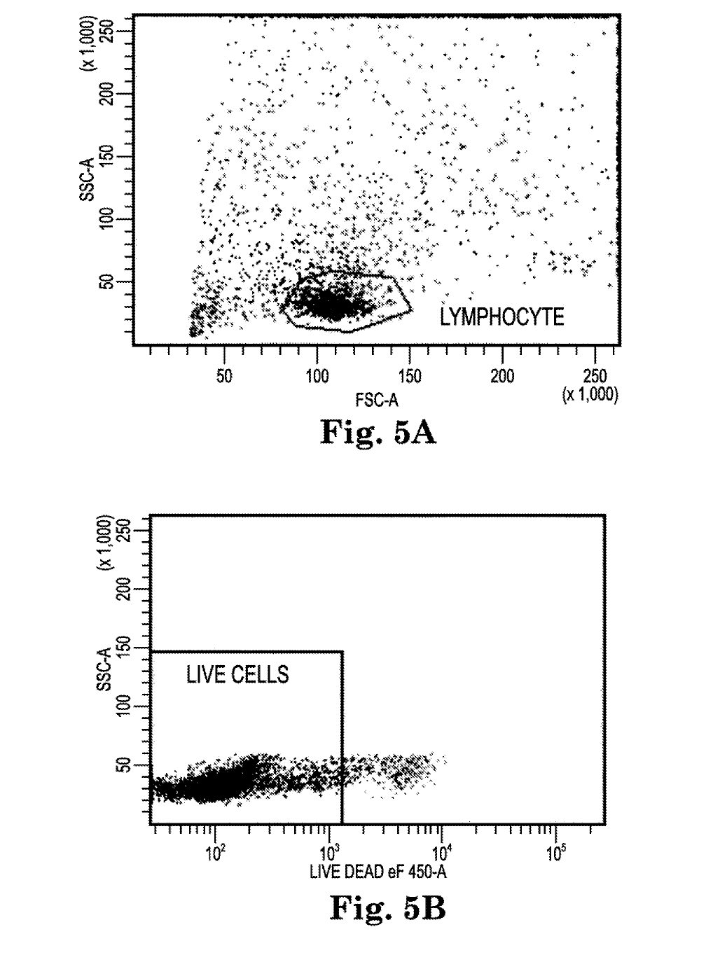

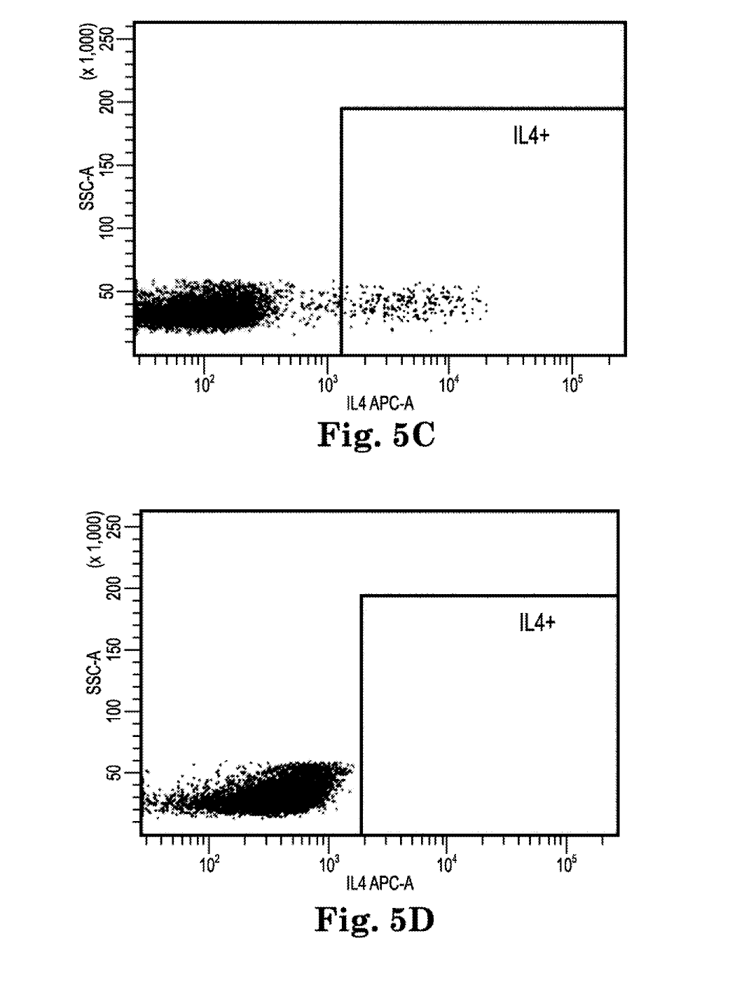

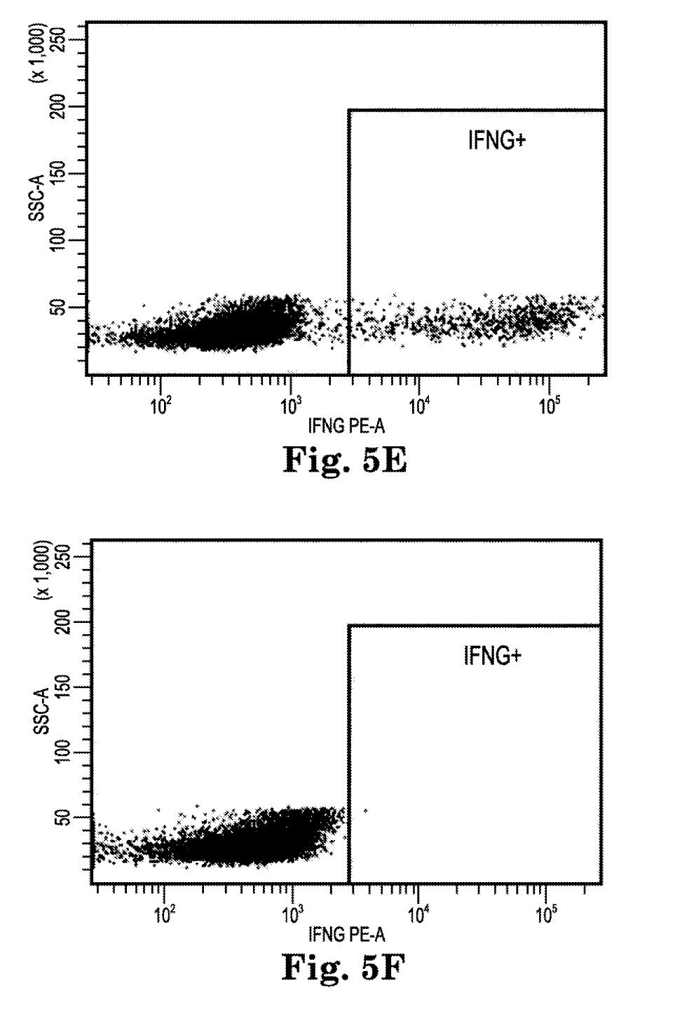

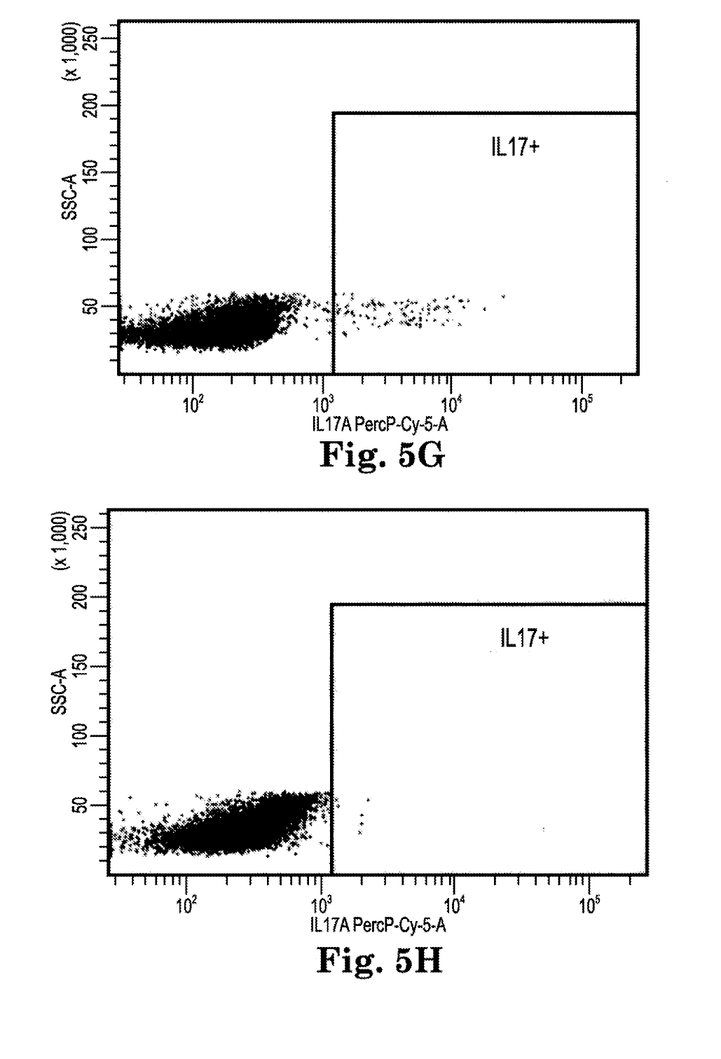

CD4 T-cells stimulated with CANDIN or CANDIN/“peptides”-treated LCs from ten subjects were stained for intracellular secretion of IFN-γ (Th1), IL-4 (Th2) and IL-17A (Th17) (FIG. 5). Increased IFN-γ secretions (>5%) were observed in CD4 T-cells exposed to CANDIN or CANDIN/“peptides”-treated LCs over media in subject 4 (9.5% and 6.9% respectively). Overall, no differences were seen in the secretion of IFN-γ, IL-4 and IL-17A between CD4 T-cells treated with LCs alone and LCs treated with CANDIN as well as between LCs alone and LCs treated with CANDIN/“peptides”.

### 4. Discussion

“Adjuvant” is derived from a Latin word, adjuvare, and means to help or to enhance. An effective vaccine adjuvant should be able to promote a strong immune response against the vaccine antigen in terms of size and durability. Antigen presenting cells (APCs) play a critical role in the initiation of immune responses. One of the desired features of an adjuvant is the ability to enhance maturation of APCs and the consequent priming of effective T-cell responses. CD40 and CD80 have been demonstrated to be critical for the activation of antigen-specific T-helper cells [31] and cytotoxic T-cells [32]. Our results have shown that the “peptides” can induce significantly higher expression of CD40 and CD80. This HPV therapeutic vaccine may be a rare vaccine in that the peptide antigens rather than the adjuvant are more able to mature APCs. These results are different from those reported by Romagnoli et al. who showed up-regulation of CD40, CD80, CD86 and HLA-DR on dendritic cells by C. albicans [33]. Since endotoxin was undetectable in “peptides”, it is unlikely that contamination may have contributed to the unexpected partial maturation effects on the LCs. We focused on examining maturation effects of LCs because our vaccine was formulated for intradermal route in order to take advantage of abundant LCs in epidermis. Studying maturation effects on other APCs such as dendritic cells and monocytes would be important in the future.

C. albicans as a component of the normal flora often colonizes the skin and the mucosal surfaces of healthy individuals. Underlying acquired immunity to C. albicans is usually present in immunocompetent individuals [34]. In this study, CANDIN and CANDIN/“peptides”, but not “peptides”, induced significant T-cell proliferation. Similar to our results, Gordon et al. demonstrated skin test positive reactions to C. albicans in 92% of healthy subjects [35], and Bauerle et al. demonstrated Candida-specific T-cell responses in 71% of healthy subjects. CANDIN is being used clinically to assess the intactness of cell-mediated immunity, so it is consistent with that that we find here that an extract from C. albicans has a T cell proliferative effect. Unfortunately, however, the maturation effects of C. albicans [33] are lost in the extract. On the other hand, it is found here that the “peptides” exert some maturation effects.

In creating this vaccine, an obstacle was encountered in being able to develop a formulation in which the “peptides” were soluble, as the E6 protein is known to be hydrophobic. While they remain soluble in acidic pH of the formulation, they are insoluble and form microparticles at a neutral pH (unpublished data). This unusual property may be contributing to the maturation effects by stimulating LCs to phagocytose these microparticles.

PRR signaling can induce APCs to express co-stimulatory molecules and cytokines necessary for activation and differentiation of T lymphocytes [37]. The cooperation of different PRRs in APCs by stimulating multiple PRRs leads to synergistic Th1 [20, 38] and cytotoxic T-lymphocyte responses [39]. C. albicans has been shown to activate many PRRs including DC-SIGN [19], dectin-1 [20], dectin-2 [21], galectin-3 [22], mannose receptor [19], mincle [40], and some TLRs [25-27, 41, 42]. Since some PRRs are increased during activation [43, 44], we investigated the presence and amplified expression of these PRRs. In this study, all PRRs examined were expressed by CANDIN and CANDIN/“peptide” pulsed LCs, and increased expressions of certain PRRs (DC-SIGN, dectin-2, mincle, monocyte receptor and TLR-9) were demonstrated in 5 of 10 subjects. Further investigations are necessary to determine which PRRs may have a role in transducing the signals from this HPV therapeutic vaccine. Dectin-1 in conjunction with TLR-2 can activate NF-KB [20], and dectin-1 can also independently mediate NFAT activation in dendritic cells leading to expression of inflammatory mediators such as IL-12p70 [45]. Therefore, it would be interesting to investigate whether CANDIN or CANDIN/“peptide” has any role in NF-_{κ}B and NFAT activation in the future.

Cytokines secreted by APCs play important roles in the process of differentiation of T-helper cells into Th1, Th2, or Th17 cells. IL-12p70 directs Th1 response while IL1β and IL6 direct the Th17 response [37, 46]. The cytokine profile in treated LCs showed IL-12p40 was the most commonly enhanced cytokine and IL-12p70 was also detected at a protein level. Published studies showed that C. albicans can induce the differentiation of specific Th1 and Th17 cells [30, 33], and Candida-specific Th1 immune responses can be detected in healthy subjects [47, 48]. These data lead us to anticipate the extract of C. albicans, CANDIN, to induce a Th1 and Th17 skewing effect. Though an increased Th1 response (IFN-γ secretion >5%) was observed in one subject, the overall results from ten subjects showed no skewing towards Th1 and Th17 responses. It may be that Candida exerts Th1 and Th17 effects through multiple mechanisms. There exist other subsets of APCs in dermis, like dermal DCs [49], which may play roles in the process of antigen presentation and T-cell activation. Furthermore, it would be important to assess the ability of this HPV therapeutic vaccine to induce HPV-specific T-cell responses. This is being investigated in the context of the ongoing clinical trial.

In summary, “peptides” (antigens) are responsible for the LC maturation effects while CANDIN (adjuvant) induces significant T-cell proliferation for this HPV therapeutic vaccine. Therefore, the antigens and the adjuvant have complementary immune enhancing effects. With time, the ongoing clinical trial will reveal whether these complementing effects will translate into effective clinical responses.

### REFERENCES FOR EXAMPLE 2

[1] Gupta R K. Aluminum compounds as vaccine adjuvants. Adv Drug Deliv Rev. 1998;32:155-72.
[2] Farhat S, Nakagawa M, Moscicki A B. Cell-mediated immune responses to human papillomavirus 16 E6 and E7 antigens as measured by interferon gamma enzyme-linked immunospot in women with cleared or persistent human papillomavirus infection. Int J Gynecol Cancer. 2009;19:508-12.
[3] Nakagawa M, Gupta S K, Coleman H N, Sellers M A, Banken J A, Greenfield W W. A favorable clinical trend is associated with CD8 T-cell immune responses to the human papillomavirus type 16 e6 antigens in women being studied for abnormal pap smear results. J Low Genit Tract Dis. 2010;14:124-9.
[4] Clifton M M, Johnson S M, Roberson P K, Kincannon J, Horn T D. Immunotherapy for recalcitrant warts in children using intralesional mumps or Candida antigens. Pediatr Dermatol. 2003;20:268-71.
[5] Horn T D, Johnson S M, Helm R M, Roberson P K. Intralesional immunotherapy of warts with mumps, Candida, and Trichophyton skin test antigens: a single-blinded, randomized, and controlled trial. Arch Dermatol. 2005;141:589-94.
[6] Johnson S M, Horn T D. Intralesional immunotherapy for warts using a combination of skin test antigens: a safe and effective therapy. J Drugs Dermatol. 2004;3:263-5.
[7] Johnson S M, Roberson P K, Horn T D. Intralesional injection of mumps or Candida skin test antigens: a novel immunotherapy for warts. Arch Dermatol. 2001;137:451-5.
[8] Kim K H, Horn T D, Pharis J, Kincannon J, Jones R, O'Bryan K, et al. Phase 1 clinical trial of intralesional injection of Candida antigen for the treatment of warts. Arch Dermatol. 2010;146:1431-3.
[9] Phillips R C, Ruhl T S, Pfenninger J L, Garber M R. Treatment of warts with Candida antigen injection. Arch Dermatol. 2000;136:1274-5.
[10] Quadrivalent vaccine against human papillomavirus to prevent high-grade cervical lesions. N Engl J Med. 2007;356:1915-27.
[11] Harper D M. Currently approved prophylactic HPV vaccines. Expert Rev Vaccines. 2009;8:1663-79.
[12] Schiller J T, Castellsague X, Villa L L, Hildesheim A. An update of prophylactic human papillomavirus L1 virus-like particle vaccine clinical trial results. Vaccine. 2008;26 Suppl 10:K53-61.
[13] Hildesheim A, Herrero R, Wacholder S, Rodriguez A C, Solomon D, Bratti M C, et al. Effect of human papillomavirus 16/18 L1 viruslike particle vaccine among young women with preexisting infection: a randomized trial. JAMA. 2007;298:743-53.
[14] Kim K H, Greenfield W W, Cannon M J, Coleman H N, Spencer H J, Nakagawa M. CD4+ T-cell response against human papillomavirus type 16 E6 protein is associated with a favorable clinical trend. Cancer Immunol Immunother. 2012;61:63-70.
[15] Nakagawa M, Stites D P, Patel S, Farhat S, Scott M, Hills N K, et al. Persistence of human papillomavirus type 16 infection is associated with lack of cytotoxic T lymphocyte response to the E6 antigens. J Infect Dis. 2000;182:595-8.

[16] Igyarto B Z, Kaplan D H. Antigen presentation by Langerhans cells. Curr Opin Immunol. 2013;25:115-9.

[17] Fahey L M, Raff A B, Da Silva D M, Kast W M. Reversal of human papillomavirus-specific T cell immune suppression through TLR agonist treatment of Langerhans cells exposed to human papillomavirus type 16. J Immunol. 2009;182:2919-28.
[18] Sato M, Sano H, Iwaki D, Kudo K, Konishi M, Takahashi H, et al. Direct binding of Toll-like receptor 2 to zymosan, and zymosan-induced NF-kappa B activation and TNF-alpha secretion are down-regulated by lung collectin surfactant protein A. J Immunol. 2003;171:417-25.
[19] Cambi A, Netea M G, Mora-Montes H M, Gow N A, Hato S V, Lowman D W, et al. Dendritic cell interaction with Candida albicans critically depends on N-linked mannan. J Biol Chem. 2008;283:20590-9.
[20] Gantner B N, Simmons R M, Canavera S J, Akira S, Underhill D M. Collaborative induction of inflammatory responses by dectin-1 and Toll-like receptor 2. J Exp Med. 2003;197:1107-17.
[21] Sato K, Yang X L, Yudate T, Chung J S, Wu J, Luby-Phelps K, et al. Dectin-2 is a pattern recognition receptor for fungi that couples with the Fc receptor gamma chain to induce innate immune responses. J Biol Chem. 2006;281:38854-66.
[22] Jouault T, El Abed-El Behi M, Martinez-Esparza M, Breuilh L, Trinel P A, Chamaillard M, et al. Specific recognition of Candida albicans by macrophages requires galectin-3 to discriminate Saccharomyces cerevisiae and needs association with TLR2 for signaling. J Immunol. 2006;177:4679-87.
[23] Bugarcic A, Hitchens K, Beckhouse A G, Wells C A, Ashman R B, Blanchard H. Human and mouse macrophage-inducible C-type lectin (Mincle) bind Candida albicans. Glycobiology. 2008;18:679-85.
[24] Netea M G, Gow N A, Munro C A, Bates S, Collins C, Ferwerda G, et al. Immune sensing of Candida albicans requires cooperative recognition of mannans and glucans by lectin and Toll-like receptors. J Clin Invest. 2006;116:1642-50.
[25] Plantinga T S, Johnson M D, Scott W K, van de Vosse E, Velez Edwards D R, Smith PB, et al. Toll-like receptor 1 polymorphisms increase susceptibility to candidemia. J Infect Dis. 2012;205:934-43.
[26] Villamon E, Gozalbo D, Roig P, O'Connor JE, Fradelizi D, Gil M L. Toll-like receptor-2 is essential in murine defenses against Candida albicans infections. Microbes Infect. 2004;6:1-7.
[27] Netea M G, Van Der Graaf C A, Vonk A G, Verschueren I, Van Der Meer J W, Kullberg B J. The role of toll-like receptor (TLR) 2 and TLR4 in the host defense against disseminated candidiasis. J Infect Dis. 2002;185:1483-9.
[28] Salvenmoser S, Seidler M J, Dalpke A, Muller F M. Effects of caspofungin, Candida albicans and Aspergillus fumigatus on toll-like receptor 9 of GM-CSF-stimulated PMNs. FEMS Immunol Med Microbiol. 2010;60:74-7.
[29] Vernal R, Leon R, Silva A, van Winkelhoff A J, Garcia-Sanz J A, Sanz M. Differential cytokine expression by human dendritic cells in response to different Porphyromonas gingivalis capsular serotypes. J Clin Periodontol. 2009;36:823-9.
[30] Zielinski C E, Mele F, Aschenbrenner D, Jarrossay D, Ronchi F, Gattorno M, et al. Pathogen-induced human TH17 cells produce IFN-gamma or IL10 and are regulated by IL-1beta. Nature. 2012;484:514-8.
[31] Schweitzer A N, Borriello F, Wong R C, Abbas A K, Sharpe A H. Role of costimulators in T cell differentiation: studies using antigen-presenting cells lacking expression of CD80 or CD86. J Immunol. 1997;158:2713-22.
[32] Bennett S R, Carbone F R, Karamalis F, Flavell R A, Miller J F, Heath W R. Help for cytotoxic-T-cell responses is mediated by CD40 signalling. Nature. 1998;393:478-80.
[33] Romagnoli G, Nisini R, Chiani P, Mariotti S, Teloni R, Cassone A, et al. The interaction of human dendritic cells with yeast and germ-tube forms of Candida albicans leads to efficient fungal processing, dendritic cell maturation, and acquisition of a Th1 response-promoting function. J Leukoc Biol. 2004;75:117-26.
[34] Romani L. Innate and adaptive immunity in Candida albicans infections and saprophytism. J Leukoc Biol. 2000;68:175-9.
[35] Gordon E H, Krouse H A, Kinney J L, Stiehm E R, Klaustermeyer W B. Delayed cutaneous hypersensitivity in normals: choice of antigens and comparison to in vitro assays of cell-mediated immunity. J Allergy Clin Immunol. 1983;72:487-94.
[36] Bauerle M, Schroppel K, Taylor B, Bergmann S, Schmitt-Haendle M, Harrer T. Analysis of the Candida albicans—specific T-cell response and oropharyngeal Candida colonization in a cohort of HIV-1-infected patients. Eur J Med Res. 2006;11:479-84.
[37] Iwasaki A, Medzhitov R. Regulation of adaptive immunity by the innate immune system. Science. 2010;327:291-5.
[38] Napolitani G, Rinaldi A, Bertoni F, Sallusto F, Lanzavecchia A. Selected Toll-like receptor agonist combinations synergistically trigger a T helper type 1-polarizing program in dendritic cells. Nat Immunol. 2005;6:769-76.
[39] Warger T, Osterloh P, Rechtsteiner G, Fassbender M, Heib V, Schmid B, et al. Synergistic activation of dendritic cells by combined Toll-like receptor ligation induces superior CTL responses in vivo. Blood. 2006;108:544-50.
[40] Wells C A, Salvage-Jones J A, Li X, Hitchens K, Butcher S, Murray R Z, et al. The macrophage-inducible C-type lectin, mincle, is an essential component of the innate immune response to Candida albicans. J Immunol. 2008;180:7404-13.
[41] Miyazato A, Nakamura K, Yamamoto N, Mora-Montes H M, Tanaka M, Abe Y, et al. Toll-like receptor 9-dependent activation of myeloid dendritic cells by Deoxynucleic acids from Candida albicans. Infect Immun. 2009;77:3056-64.
[42] Netea M G, van de Veerdonk F, Verschueren I, van der Meer J W, Kullberg B J. Role of TLR1 and TLR6 in the host defense against disseminated candidiasis. FEMS Immunol Med Microbiol. 2008;52:118-23.
[43] Biswas I, Garg I, Singh B, Khan G A. A key role of toll-like receptor 3 in tissue factor activation through extracellular signal regulated kinase 1/2 pathway in a murine hypoxia model. Blood Cells Mol Dis. 2012;49:92-101.
[44] Sinha S, Guo Y, Thet S, Yuan D. IFN type I and type II independent enhancement of B cell TLR7 expression by natural killer cells. J Leukoc Biol. 2012;92:713-22.
[45] Goodridge H S, Simmons R M, Underhill D M. Dectin-1 stimulation by Candida albicans yeast or zymosan triggers NFAT activation in macrophages and dendritic cells. J Immunol. 2007;178:3107-15.
[46] Zhou L, Chong M M, Littman D R. Plasticity of CD4+ T cell lineage differentiation. Immunity. 2009;30:646-55.

[47] La Sala A, Urbani F, Torosantucci A, Cassone A, Ausiello C M. Mannoproteins from Candida albicans elicit a Th-type-1 cytokine profile in human Candida specific long-term T cell cultures. J Biol Regul Homeost Agents. 1996;10:8-12.

[48] Nisini R, Romagnoli G, Gomez M J, La Valle R, Torosantucci A, Mariotti S, et al. Antigenic properties and processing requirements of 65-kilodalton mannoprotein, a major antigen target of anti-Candida human T-cell response, as disclosed by specific human T-cell clones. Infect Immun. 2001;69:3728-36.
[49] Valladeau J, Saeland S. Cutaneous dendritic cells. Semin Immunol. 2005;17:273-83.

### Example 3

A Phase I Dose-Escalation Clinical Trial of a Peptide-Based Human Papillomavirus Therapeutic Vaccine with Candida Skin Test Reagent as a Novel Vaccine Adjuvant for Treating Women with Biopsy-Proven Cervical Intraepithelial Neoplasia 2/3

### Abstract

### Purpose

Non-surgical treatments for cervical intraepithelial neoplasia 2/3 (CIN2/3) are needed as surgical treatments have been shown to double preterm delivery rate. The goal of this study was to demonstrate safety of a human papillomavirus (HPV) therapeutic vaccine called PepCan, which consists of four current good manufacturing production-grade peptides covering the HPV type 16 E6 protein and Candida skin test reagent as a novel adjuvant.

### Patients and Methods

The study was a single-arm, single-institution, dose-escalation Phase I clinical trial, and the patients (n=24) were women with biopsy-proven CIN2/3. Four injections were administered intradermally every 3 weeks in limbs. Loop electrical excision procedure was performed 12 weeks after the last injection for treatment and histological analysis. Six subjects each were enrolled (50, 100, 250, and 500 ug per peptide).

### Results

The most common adverse events were injection site reactions, and none of the patients experienced dose-limiting toxicities. The best histological response was seen at the 50 ug dose level with a regression rate of 83% (n=6), and the overall rate was 52% (n=23). Vaccine-induced immune responses to E6 were detected in 65% of recipients (significantly in 43%). Systemic T-helper type 1 (Th1) cells were significantly increased after 4 vaccinations (p=0.02).

### Conclusion

This study demonstrated that PepCan is safe. A significantly increased systemic level of Th1 cells suggests that Candida, which induces interleukin-12 in vitro, may have a Th1 promoting effect. A Phase II clinical trial to assess the full effect of this vaccine is warranted.

### LIST OF ABBREVIATIONS AND ACRONYMS

AE, adverse event

CIN 2/3, cervical intraepithelial neoplasia 2/3

ELISPOT, enzyme-linked immunospot

HPV, human papillomavirus

IL-12, interleukin 12

LEEP, loop electrical excision procedure

PBMC, peripheral blood mononuclear cell

Th1, T-helper type 1

Th2, T-helper type 2

Treg, regulatory T-cell

### INTRODUCTION

Cervical intraepithelial neoplasia 2/3 (CIN2/3) is a precursor of cervical cancer which is the fourth most common cancer among women globally despite availabilities of effective screening tests and prophylactic vaccines. The annual global incidence of cervical cancer is 528,000 cases and the mortality is 266,000 cases.¹ It is almost always caused by human papillomavirus (HPV). HPV causes not only cervical cancer, but also anal, oropharyngeal, penile, vaginal, and vulvar cancers; it is estimated to be responsible for 5.2% of cancer cases in the world.^{2, 3}

Standard surgical treatments of CIN2/3 such as loop electrical excision procedure (LEEP) are effective but result in doubling of preterm delivery rate from 4.4% to 8.9%.^{4, 5} Therefore, the new treatment guidelines published in 2013 recommend 1-2 years of close observation in women, with cervical intraepithelial neoplasia 2, who are less than 25 years in age or who plan to have children at any age. For cervical intraepithelial neoplasia 3, treatment is recommended but observation is an accepted option.⁵ Non-surgical alternatives which would leave the cervix anatomically intact are needed but not currently available. When approved, an HPV therapeutic vaccine is likely to become the first-line therapy for treating CIN2/3 in young women. Furthermore, an HPV therapeutic vaccine, which requires only injections, could benefit women in developing regions where surgical expertise to perform excisional procedures may not be available.

HPV transformation of squamous epithelium to a malignant phenotype is mediated by two early gene products, E6 and E7,⁶ and their expression is necessary for HPV type 16 transformation of human cells.^{7, 8} T-cell responses to HPV type 16 E6 protein have been associated with favorable clinical outcomes such as viral clearance⁹ and regression of cervical lesions.^{10, 11} The E6 protein is an especially attractive target for immunotherapy since it is a viral protein, and attacking self-protein (i.e., autoimmunity) is not of concern.

Traditionally, recall antigens, which typically include a panel of Candida, mumps, and Trichophyton, were used as controls to indicate intact cellular immunity when patients were being tested for Tuberculosis by placement of PPD intradermally. T-cell mediated inflammation would become evident in 24-48 hours.¹² A number of studies have demonstrated that recall antigen injections can also be used to treat common warts (a condition also caused by HPV), and several studies have shown that treating warts with recall antigens is effective not only for injected warts but also distant untreated warts.¹³⁻¹⁶ This suggests that T-cells may have a role in wart regression. In a recently completed Phase I investigational new drug study (NCT00569231) in which the largest wart was treated with Candida, complete resolution of the treated warts was reported in 82% (nine of 11) of patients.¹⁶ Furthermore, T-cell responses to the HPV 57 L1 peptide were detected in 67% (six of nine) of the complete responders.¹⁶ These immune-enhancing and possible anti-HPV effects of Candida prompted the use of Candida as a vaccine adjuvant. Safety, efficacy, and immune responses of PepCan have been evaluated in a Phase I clinical trial (NCT01653249).

### RESULTS

### Safety

Patient characteristics and adverse events (AEs) are summarized in Tables 3 and 4 respectively. None of the vaccine recipients experienced any dose-limiting toxicity, and the most frequent AEs were immediate (seen with all injections) and delayed injection site reactions. More grade 2 immediate and delayed injection site reactions were recorded at the higher doses [odds ratio of 33.0 (2.9, 374.3), p<0.0001 for the immediate reaction and odds ratio of 4.5 (0.9, 23.8), p=0.07 for the delayed reaction]. No patients discontinued due to AEs.

### Efficacy

CIN2/3 lesions are usually asymptomatic so vaccine response was assessed by histological regression. CIN2/3 was no longer present at exit in 9 of 23 (39%) patients who completed the study (Table 3), the remaining CIN2/3 lesions measured ≦0.2 mm² in 3 (13%) patients. The histological response rates by dose were 83%, 50%, 33%, and 40% with the best response at the lowest dose. None progressed to cervical squamous cell carcinoma. The regression rates were similar for CIN2 (50%) and CIN3 (62%), and in CIN2/3 associated and not associated with HPV 16 (44% vs. 57%). The mean number of cervical quadrants with visible lesions decreased significantly from 2.1±1.1 (range 0 to 4) quadrants prior to vaccination 0.8±1.0 (0 to 3) quadrants after vaccination (p<0.0001). However, five of the 12 subjects with no visible lesions after vaccination were histological non-responders with persistent CIN2/3. At least one HPV type present at entry became undetectable in 13 of 23 (57%) patients. By dose, the rates were 83%, 50%, 50%, and 40% with the highest undetectability at the lowest dose.

### Immune Responses

New CD3 T-cell responses to at least one region of the E6 protein were detected in 15 of 23 patients (65%, Table 3) with the increased responses after vaccination being statistically significant in 10 patients (43%). The CD3 T-cell response rates to E6 by dose were 83%, 67%, 83%, and 20% with the best responses at the 50 and 250 ug doses. The percentages of statistically significant increase in E6 responses were 50%, 50%, 50%, and 20% by dose. Patients 4 and 11 demonstrated statistically significant increases in one of the regions of E7 likely representing epitope spreading.

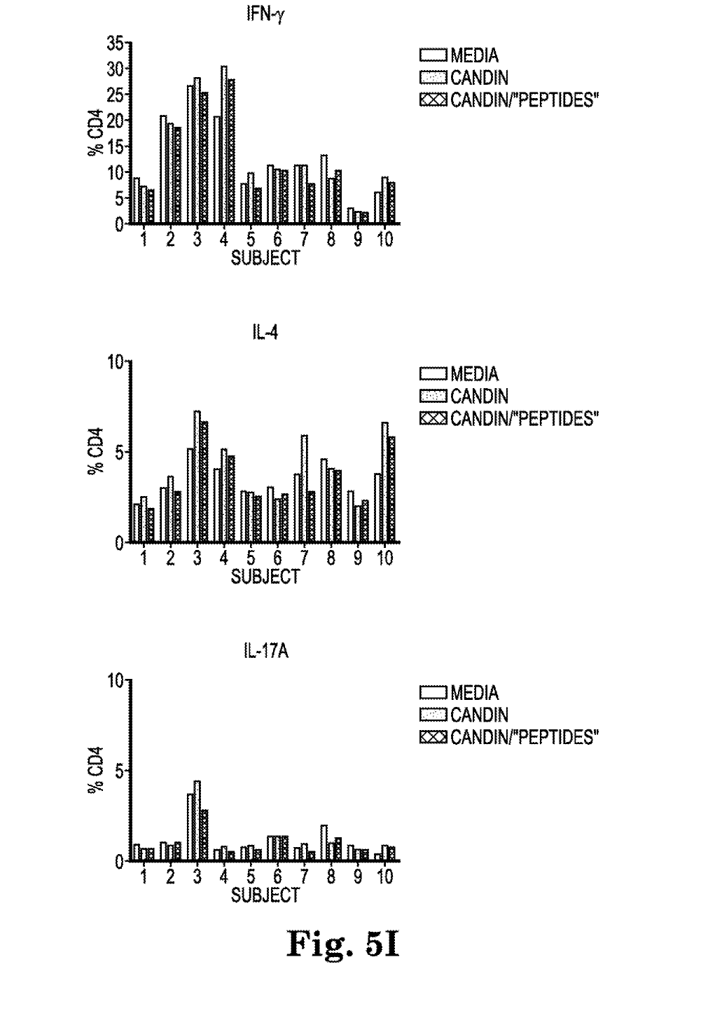

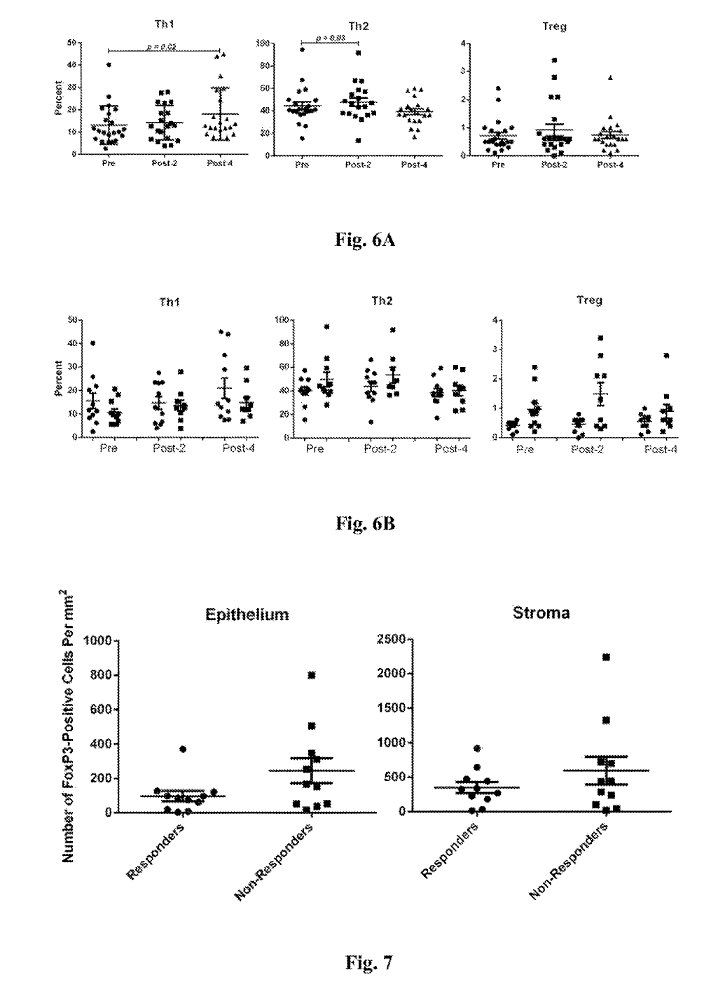

The percentages of regulatory T-cells (Tregs) were not changed after vaccinations while those of T-helper type 1 (Th1) cells were significantly increased (p=0.02). The percentages of T-helper type 2 (Th2) cells increased significantly initially after 2 vaccinations (p=0.03), but decreased below the baseline after 4 vaccinations (FIG. 6A). The differences between the responders and non-responders approached significance for Tregs at baseline (p=0.07) and at post-2 vaccinations (p=0.08, FIG. 6B). The number of Tregs infiltrating lesional cervical epithelium and the underlying stroma was lower in histological responders compared to non-responders, and approached statistical significance for the epithelium (p=0.08, FIG. 7).

### Medicinal Product

Precipitates became visible immediately at the 250 ug peptide dose-equivalent, and at other peptide dose-equivalents at 20 minutes. For peptides combined with Candida (CANDIN, Nielsen Biosciences, Inc., #59584-138-01) the precipitates formed at 20 minutes for the 500 ug peptide dose-equivalent, at 40 minutes for the 100 and 250 ug peptide dose-equivalents, and at 80 minutes for the 500 ug peptide dose-equivalent.

### HLA

Compared to the general population in the United States, HLA frequencies for A30, A33, A66, B14, B15, B40, C03, C18, DQ03, DQ05, and DR03 were significantly increased in patients who received vaccination (n=24). In order to eliminate the effect of disparate racial distributions between these two populations, expected HLA frequencies were calculated based on the racial distribution of the patients. Significant increases were observed in the patients for A32, B14, B15, B35, B40, C03, DQ03, and DR03. When the HLA frequencies were compared between histological responders and non-responders, B44 was significantly higher in responders (4 of 24 genes) compared to non-responders (0 of 22 genes, p=0.04).

TABLE 3
Subject characteristics, HPV types, T-cell response, and histological diagnoses at exit.

| | | | | | | Exit |
|---|---|---|---|---|---|---|
| Dose | No | Age | Race | HPV types at entry* | CD 3 T-cell responses in E6 detected after vaccination\hat{ } | histology |
| 50 μg | 1 | 36 | Caucasian | 16, 52, 84 | None | CIN2,3 |
| | 2 | 49 | Caucasian | 45, 84 | 46-70 | CIN3^{#} |
| | 3 | 28 | Caucasian | 66, 84 | 16-40; 46-70 | No CIN |
| | 4 | 42 | African American | 45 | 1-25; 31-55; 46-70; 61-85; 76-100; 91-115; 106-130; 121-145 | CIN1 |
| | 5 | 31 | African American | 52, 53 61 | 16-40, 76-100; 91-115 | No CIN |
| | 6 | 41 | Caucasian | 16, 31, 58 | 1-25; 91-115; 136-158 | No CIN |
| 100 μg | 7 | 28 | African American | 26, 33, 51, 55, 58, 81 | 31-55; 106-130; 121-145; 136-158 | No CIN |
| | 8 | 22 | African American | 45, 56 | None | No CIN |
| | 9 | 34 | African American | 16 | 121-145; 136-158 | CIN2,3 |
| | 10 | 31 | African American | 35, 72, 83 | 16-40; 121-145; 136-158 | CIN2,3 |
| | 11 | 28 | African American | 16 | 1-25; 16-40; 31-55; 46-70; 61-85; 76-100; 91-115; 121-145; | CIN2 |
| | | | | | 136-158 | |
| | 12 | 32 | Mixed | 16 | None | No CIN |
| 250 μg | 13 | 29 | African American | 39, 73, IS39 | 106-130 | CIN2,3 |
| | 14 | 31 | African American | 58 | None | CIN2^{#} |
| | 15 | 32 | African American | 35 | 1-25 | CIN3 |
| | 16 | 25 | Caucasian | 16 | 16-40; 31-55; 46-70; 76-100; 91-115; 136-158 | CIN3 |
| | 17 | 22 | African American | 35, 59, 66, 81, | 1-25; 16-40; 46-70; 61-85; 76-100; 106-130; 121-145; 136-158 | CIN1 |
| | | | | CP6108 | | |
| | 18 | 23 | Caucasian | 45, 52, 62, 82 | 1-25; 31-55; 46-70; 61-85; 76-100; 91-115 | CIN3 |
| 500 μg | 19 | 29 | Caucasian | 16, 53 | 61-85; 91-115; 121-145 | CIN2,3 |
| | 20 | 26 | Caucasian | 16, 35, 58, 66 | None | CIN3^{#} |
| | 21 | 23 | African American | 58 | None | CIN3 |
| | 22 | 27 | Caucasian | 6, 52, 66, CP6108 | None | CIN2 |
| | 23 | 26 | African American | 31, 35 | NA | NA |
| | 24 | 32 | Caucasian | 16, 62 | None | No CIN |

*HPV types which became undetectable after vaccinations are shown in italics, and persistent HPV types are shown in bold.
\hat{ }CD3 T-cell response (positivity index ≧2.0 as long as at least 80 per 10⁶ IFN-g secreting CD3 cells detected) in new E6 region(s) after vaccinations.
^{#}considered to be a partial responder as the area of CIN3 measured ≦2 mm²
NA = not applicable

TABLE 4A
Summary of adverse events

| | CTCAE Grade, Number of Events | | | | | | | |
|---|---|---|---|---|---|---|---|---|
| | (Number of Patients) | | | | | | | |
| | Grade 1 | | | | Grade 2 | | | |
| Dose (ug/peptide) | 50 | 100 | 250 | 500 | 50 | 100 | 250 | 500 |
| Adverse event | | | | | | | | |
| Injection site | 23(6) | 24(6) | 18(6) | 11(6) | 1(1) | | 6(3) | 11(6) |
| reaction, | | | | | | | | |
| immediate^{a} | | | | | | | | |
| Injection site | 5(4) | 4(3) | 3(3) | 4(3) | 1(1) | 1(1) | 3(1) | 5(4) |
| reaction, other, | | | | | | | | |
| delayed^{b} | | | | | | | | |
| Myalgia | 8(3) | 4(1) | 4(1) | 4(3) | | | | 1(1) |
| Fatigue | 5(3) | 1(1) | 2(1) | 2(2) | | 1(1) | | |
| Diarrhea | 1(1) | | | | | | | |
| Nausea | 2(2) | 5(3) | | 5(4) | | | | |
| Vomiting | | | | 1(1) | | | | |
| Headache | 3(2) | 3(3) | 5(2) | 6(2) | | | | 2(1) |
| Pain-body | 2(2) | | | | 1(1) | | | 2(1) |
| Alopecia | | | | | 1(1) | | | |
| Feverish^{c} | 1(1) | 2(1) | 1(1) | 1(1) | | | | |
| Hot flashes | | | | 1(1) | | | | |
| Muscle spasm | 1(1) | | | | | | | |
| Flu-like symptoms | 4(1) | 3(1) | | | | | 1(1) | |
| Photophobia | | | | 1(1) | | | | |
| Agitation | | | 1(1) | 1(1) | | | | |
| Vertigo | | | 1(1) | | | | | |
| Dizziness | | | | 1(1) | | | | |
| Neutropenia | 1(1) | | | | | | | |
| Hypokalmia | 4(4) | 1(1) | 2(2) | 1(1) | | | | 1(1) |
| Thrombocytopenia | | | | 1(1) | 1(1) | | | |
| GGT increased | | 1(1) | | | | | | |

^{a}appearing <24 hours from time of vaccination;
^{b}appearing >24 hours from time of vaccination;
^{c}feeling warm without evidence of temperature >38.0° C.

TABLE 4B
Detailed descriptions of injection site reactions

| | CTCAE Grade, Number of Events, | | | | | | | |
|---|---|---|---|---|---|---|---|---|
| | (Number of patients) | | | | | | | |
| Dose | Grade 1 | | | | Grade 2 | | | |
| (ug/peptide) | 50 | 100 | 250 | 500 | 50 | 100 | 250 | 500 |
| Adverse Event | | | | | | | | |
| Injection site | 23(6) | 24(6) | 18(6 | 11(6) | 1(1) | | 6(3) | 11(6) |
| reaction, | | | | | | | | |
| immediate | | | | | | | | |
| Pain | | | | | 1(1) | | 6(3) | 11(6) |
| Redness | 24(6) | 23(6) | 24(6) | 22(6) | | | | |
| Swelling | 2(1) | 7(2) | 1(1) | 8(4) | | | | |
| Welt | 7(4) | 16(5) | 22(6) | 21(6) | | | | |
| Tenderness | | | | 1(1) | | | | |
| Itching | 13(5) | 13(5) | 11(5) | 9(4) | | | | |
| Burning | | 1(1) | 1(1) | 1(1) | | | | |
| Warmness | | | 1(1) | 1(1) | | | | |
| Injection site | 5(4) | 4(3) | 3(3) | 4(3) | 1(1) | 1(1) | 3(1) | 5(4) |
| reaction, | | | | | | | | |
| delayed | | | | | | | | |
| Pain | | | | | 1(1) | 1(1) | 3(1) | 5(4) |
| Redness | 5(4) | 2(2) | 5(3) | 3(3) | | | | |
| Swelling | 5(4) | 2(2) | 2(2) | 5(5) | | | | |
| Welt | | | | | | | | |
| Tenderness | | | | | | | | |
| Itching | 1(1) | 2(2) | 3(3) | 4(4) | | | | |
| Burning | | 2(1) | | | | | | |
| Warmness | | | | 1(1) | | | | |

### DISCUSSION

The safety of this HPV therapeutic vaccine has been demonstrated as no dose-limiting toxicities were reported. The most common AEs were immediate injection site reactions which were reported with all vaccinations. In contrast, only very rare observations of immediate reactions were recorded when Candida alone was injected for treating common warts.¹⁶ Therefore, the peptides are likely to be the culprit. These AEs may be related to the peptides' property of forming microparticles when placed in a neutral pH, although they are stably soluble in its formulation which has pH of 4. These microparticles would likely enhance the immunogenicity of the vaccine as they may stimulate Langerhans cells to phagocytose them.^{∫}The unexpected AEs were delayed injection site reactions, which were defined as occurring equal to or more than 24 hours after injections. However, they appeared from 1 to 6 days afterwards and therefore not all of them could be dismissed as delayed-type hypersensitivity reactions.¹² The timing of occurring several days afterwards raises a possibility of de novo immune responses occurring at the site.¹⁸

The best histological regression rate was recorded with the 50 ug group (83%) while the overall regression rate was 52%. Both rates were higher than the 22% regression rate reported for a historical placebo group in another clinical trial of HPV therapeutic vaccine with a similar study design.¹⁹ Kenter et al. reported the complete histological regression rate of 25% at 3 months and 47% at 12 months in patients with HPV 16-positive high-grade vulvar intraepithelial lesions who received another peptide-based HPV therapeutic vaccine.²⁰ Therefore, the vaccine response is expected to increase with the extended observation period of 12 months which is being planned for the Phase II clinical trial.

New HPV 16 E6-specific CD3 T-cell responses were observed in 65% of patients and more than half had statistically significant increases, attesting to the immunogenicity of PepCan. Others have reported significant correlations between HPV therapeutic vaccine-induced immune responses and clinical outcomes.^{20, 21} Kenter et al. reported significantly higher numbers of interferon-γ producing CD4 T-cells and stronger proliferative responses in patients with complete responses compared to those with no responses at 3 months.²⁰ In a clinical trial of imiquimod and HPV therapeutic vaccination treating vulvar intraepithelial lesions, Daayana et al. found significantly increased lymphocyte proliferation to the HPV vaccine antigens in responders.²¹ We found no significant association between CD3 T-cell responses and histological regression as five responders had no new responses against E6. This may be due to a limitation of peripheral detection as HPV-specific T-cells would eventually need to reach the cervix to carry out their anti-HPV activity.

Epitope spreading is a process in which antigenic epitopes distinct from and non-cross-reactive with an inducing epitope become additional targets of an ongoing immune response, and it has been associated with favorable clinical outcomes for cancer immunotherapy.²² Two vaccine recipients demonstrated significant increases in T-cell response to HPV type 16 E7 protein in addition to the E6 protein contained in the vaccine. One had persistent HPV type 16 infection, and the other one had persistent HPV type 45 infection. As there is little amino acid homology between the E7 proteins of HPV types 16 and 45, this patient may have had a latent HPV type 16 infection undetectable by the PCR method or may have had a reactivation of memory T-cell response from her past HPV type 16 infection. HPV 16 is the most common HPV type detected,²³⁻²⁷ and a lifetime risk of acquiring HPV 16 is estimated to be 50%.²⁸

As an investigational adjuvant, granulocyte monocyte colony-stimulating factor has been reported to inadvertently increase Tregs resulting in less effective vaccine response.²⁹ Therefore, we monitored levels of Tregs, which were minimally changed. Th1 cells were significantly increased, supporting the immunostimulatory effect of PepCan. Our earlier work showed that Candida has T-cell proliferative effects, and that the cytokine most frequently produced by Langerhans cells exposed to Candida was interleukin-12 (IL-12).^{17, 30} Therefore, Candida is likely responsible for the increased levels of Th1 cells after vaccination, and may be an effective vaccine adjuvant for other therapies designed to promote T-cell activity, not only for other pathogenic antigens but also for tumor antigens in new cancer immunotherapies. Th1 polarization of T helper cells by IL-12 has been demonstrated previously in vitro³¹ and in a murine mode1.³² However, this is the first example, to our knowledge, of Th1 promotion due to an agent that likely induces IL-12 secretion in vivo. IL-12 is also known to be a potent inducer of antitumor activity.³³ Given the demonstrated safety profile of PepCan, this may be an effective alternative to systemic administration of IL-12 with which toxicities have been problematic.³³ Although Treg levels were not increased after vaccination, they may have an effect on whether subjects would respond to the vaccine, as pre-vaccination Treg levels were lower in non-responders compared to responders, though not significantly. This difference persisted over time. Therefore, it is possible that some pretreatment to decrease Treg levels prior to vaccine initiation such as administration of cyclophosphamide^{34, 35} may improve vaccine response. Treg levels were also higher in non-responders compared to responders in the cervical lesions and the underlying stroma (though the differences were not statistically significant) possibly supporting the negative role of Treg in vaccine response.

HLA gene frequencies of B14, B15, B40, C03, DQ03, and DR03 molecules were significantly higher in our patients compared to the general population in the United States and the general population adjusted for the racial distribution of the patients. Increased risk of cervical neoplasia associated with DQ03 has been reported by others.³⁶⁻³⁸ When histological responders and non-responders were compared only B44 was significantly elevated in responders compared to non-responders. This implies that the B44 molecule may present effective epitopes of HPV 16 E6 protein. However, no such epitopes have been described to date to our knowledge.

Unexpectedly, histological regression, undetectability of at least one HPV type present at entry, and immune responses were all superior at the lowest dose compared to the highest dose, and we plan to use the lowest dose for the Phase II clinical trial. As the number of subjects in each dose group was small (n=6), this study was not powered to show significant differences. As no patient with percent Treg equal to greater than 0.8% prior to vaccination responded, it is possible that the higher prevaccination Treg levels at higher doses may have influenced the outcome. The median percentages of Tregs were 0.5, 0.4, 0.7 and 0.9 by dose respectively. Nevertheless, we have shown that PepCan is safe and well tolerated, and a Phase II clinical trial in which the observation period is extended to 12 months for maximal response is warranted.

### PATIENTS AND METHODS

### Patients

This clinical trial was a Phase I single-arm, single-site, dose escalation study. Patients (n=37) were enrolled between Sep. 2012 and Mar. 2014, and those with biopsy-proven CIN2/3 (n=24) were eligible for vaccination (Table 3).

Vaccination was started within 60 days of biopsy date, and 4 injections were given 3 weeks apart. Each patient received the same dose of the peptides, and 6 subjects each were recruited in each dose group.

At the screening visit, the cervix was visualized under a colposcope after applying acetic acid, biopsies were obtained, Thin-Prep (Hologic, #70097-0001) was collected for HPV-DNA testing (Linear Array HPV Genotyping Test, Roche Molecular Diagnostics, #04472209190 and #03378012190), and routine laboratory testing was performed (complete blood count, sodium, potassium, chloride, carbon dioxide, blood urea nitrogen, creatinine, aspartate transaminase, alanine transaminase, lactate dehydrogenase, γ-glutamyl transpeptidase, total bilirubin, and direct bilirubin). Patients who already were diagnosed with biopsy-proven CIN2/3 were also eligible as long as the first vaccine injection could be given within 60 days, and other inclusion criteria were met (ages 18 to 50 years old, blood pressure ≦200/120 mm Hg, heart rate 50 to 120 beats per minute, respiration ≦25 breaths per minute, temperature ≦100.4° F., white count ≧3×10⁹/L, hemoglobin ≧8 g/dL, and platelet count ≧50×10⁹/L). Being positive for HPV 16 was not required due to possible cross-protection^{10, 11, 39, 40} and de novo immune stimulation.^{14, 16} Exclusion criteria included a history of disease or treatment causing immunosuppression, pregnancy, breast feeding, allergy to Candida, a history of severe asthma, current use of beta-blocker, and a history of invasive squamous cell carcinoma of the cervix. Urine pregnancy test was performed prior to each injection, and blood was drawn for routine laboratory testing and immunological assessments immediately prior to the first and third injections. The vaccine was administered intradermally in any limb. Twelve weeks after the last injection, blood was drawn, ThinPrep sample was collected, and LEEP was performed. Safety and tolerability were assessed from the time informed consent was obtained until the day LEEP was performed using version 4.1 of the National Cancer Institute Common Terminology Criteria for Adverse Events. Dose-limiting toxicities were defined as vaccine-related allergic and autoimmune AEs greater than grade 1 and any other AEs greater than grade 2. Efficacy was based on histological grading of the LEEP samples. A patient with no dysplasia or CIN 1 was considered to be a complete responder, and a patient with CIN2/3 measuring ≦0.2 mm² was considered to be a partial responder. The study was approved by the Institutional Review Board, and a written informed consent was obtained from each participant.

### Vaccine Composition

The vaccine consisted of four current good manufacturing production-grade synthetic peptides covering the HPV 16 E6 protein with the following sequences:

### E6 1-45 (Ac-MHQKRTAMFQDPQERPRKLPQLCTELQTTIHDIILECVYCKQQLL-NH2) (SEQ ID NO:10),

### E6 46-80 (Ac-RREVYDFAFRDLCIVYRDGNPYAVCDKCLKFYSKI-NH2) (SEQ ID NO:11),

### E6 81-115 (Ac-SEYRHYCYSLYGTTLEQQYNKPLCDLLIRCINCQK-NH2) (SEQ ID NO:12), and

E6 116-158 (Ac-PLCPEEKQRHLDKKQRFHNIRGRWTGRCMSCCRSSRTRRETQL-NH2) (SEQ ID NO:13).¹⁷ The two regions (amino acids 46-70 and 91-115) previously shown to be most immunogenic in terms of CD8 T-cell responses were preserved.¹¹

Reconstituted peptides alone or reconstituted peptides with Candida, at the same proportions as in the four doses being tested (but one sixth in total volume), were combined with RPMI1640 media (Mediatech, Inc., #10-040-CV) with 10% fetal calf serum (Atlanta Biologicals, #S11150H) in a 24 well plate. A total volume for each condition was 1 ml. The mixtures were incubated at 37° C. with 5% carbon dioxide. Visual inspection to detect precipitate formation was performed every 20 minutes for the first 80 minutes, and every 40 minutes for the following 160 minutes. Photomicrographs were taken at 24 hours using AxioCam Mrc5 attached to Axiolmager Z1 with Axio Vision software (Carl Zeiss AG) in the University of Arkansas for Medical Sciences Digital Microscopy Laboratory.

Prior to injecting patients, lyopholized peptides were reconstituted with sterile water and were mixed with 300 ul of Candida albicans skin test reagent (CANDIN) in a syringe. The amount of peptide per injection was 50, 100, 250, or 500 ug per peptide, and the total injection volume was 0.4, 0.5, 0.75, or 1.2 ml respectively.

### Immunological Assessments

### Peripheral HPV 16-Specific T-Cell Responses (also see Supplementary Appendix)

T-cell lines were established from three blood draws from each patient as described previously with minor modifications.^{10, 11, 41} In short, peripheral blood mononuclear cells (PBMCs) were isolated from heparinized whole blood using a Ficoll density-gradient centrifugation method, separated into CD14+ monocytes and CD14-depleted PBMCs, and cryopreserved. Autologous dendritic cells were established by growing monocytes in the presence of granulocyte monocyte-colony stimulating factor (50 ng/mL, Sanofi-Aventis, #420039) and recombinant interleukin-4 (100 U/mL, R&D Systems, #204-IL-050) for seven days, and were matured by 48-hour culture in wells containing irradiated mouse L-cells expressing CD40 ligands. CD3 T-cells were magnetically selected (Pan T Cell Isolation Kit II, Miltenyi Biotec, #130-096-535) from CD14-depleted PBMCs. HPV 16 E6- and E7-specific CD3 T-cell lines were established by in vitro stimulation of CD3 cells for seven days with autologous dendritic cells pulsed with E6-glutathione S-transferase and E6 expressing recombinant vaccinia virus or E7-glutathione S-transferase and E7 expressing recombinant vaccinia virus.^{10, 11, 41-43} In vitro stimulation was repeated for an additional seven days.

ELISPOT assays were performed in triplicate using overlapping peptides covering the E6 and E7 proteins of HPV 16, as described.⁴¹ MultiScreen-MAHA plates (Millipore, #MAHAS4510) were coated with mouse anti-human interferon-gamma monoclonal primary antibody (5 ug/mL, 1-D1K, Mabtech, #3420-3-1000). The coated plates were washed and blocked. After incubating at 37° C. for 1 hour, 2.5×10⁴ CD3+ cells per well were added, along with pools of peptides (10 uM each) in triplicate. Negative control wells contained medium only, and positive control wells contained phytohaemagglutinin at 10 ug/mL(Remel, #R30852801). Following a 24 hour incubation, the plates were washed and a secondary antibody was added (1 ug/mL of biotin-conjugated anti-IFN-γ monoclonal antibody; 7-B6-1, Mabtech, #3420-6-250). After a 2 hour incubation and washing, avidin-bound biotinylated horseradish peroxidase (Vectastain ABC Kit, Vector Laboratories, #PK-6100) was added. After 1 hour of incubation, the plates were washed, and stable diaminobenzene (50 uL, Life Technologies, #750118) was added. After developing the reaction for 5 minutes, the plates were washed with deionized water. Spot-forming units were be counted by an automated ELISPOT analyzer (AID ELISPOT Classic Reader; Autoimmun Diagnostika GmbH). An HPV-specific T-lymphocyte response was considered to be positive if spot-forming units in peptide containing wells were at least two times higher than in the corresponding negative-control wells (i.e., positivity index of ≧2.0),⁴⁴ and if at least 80 spot-forming units per 10⁶ CD3 T-cells were present in peptide containing wells. If any region was found to be positive after 2 or 4 vaccinations, and the positivity index was higher than that at the baseline, the number of peptide-specific spot forming units for each well was calculated by subtracting the number of background spot forming units from the negative control wells containing media only. Paired t-test was used to assess the significance of differences after 2 or 4 vaccinations compared to the baseline.

### Peripheral Immune Cells

Thawed PBMCs were stained with relevant isotype controls and combinations of monoclonal antibodies to analyze Th1, Th2, and Tregs: fluorescein isothiocyanate-labeled anti-human CD4 (clone RPA-T4, eBioscience, #45-0048-41), phycoerythrin-labeled anti-human/mouse T-bet (clone 4B10, eBioscience, #12-5825-82), PerCP-Cy5.5-labeled anti-human CD25 (clone BC96, eBioscience, #45-0259-42), allophycocyanin-labeled anti-human Foxp3 (clone PCH101, eBioscience, #17-4776-42), and phycoerythrin -Cy7 labeled anti-human/mouse GATA3 (clone L50-823, Becton Dickinson Biosciences, #560405). Cells were first stained with antibodies for surface markers CD3, CD4, and CD25. Staining for intracellular T-bet, GATA3, and Foxp3 was performed using the Foxp3 staining kit (eBioscience, #00-5523-00) according to the manufacturer's instructions. Flow cytometric analysis was performed with FACS Fortessa using FACS Diva software (Becton Dickinson Biosciences) in the University of Arkansas for Medical Sciences Microbiology and Immunology Flow Cytometry Core Laboratory. Ten thousand events were acquired in the lymphocyte gate. CD4 cells were expressed as a percentage of lymphocytes, Th1 cells were expressed as a percentage of CD4 cells positive for Tbet, Th2 cells were expressed as a percentage of CD4 cells positive for GATA3, and regulatory T-cells were expressed as a percentage of CD4 cells positive for CD25 and Foxp3.¹⁰

### Cervical Regulatory T-Cells

Nuclear localization of FoxP3 was utilized to quantitate Tregs using a digital pathology system.^{45, 46} Slides of LEEP samples were pretreated with a target retrieval solution (Dako Corporation, #S2369), peroxidase block (Dako Corporation, #S2003), and serum-free protein block (Dako Corporation, #X0909) prior to performing immunohistochemistry with primary goat anti-human polyclonal antibody against FoxP3 (R&D Systems, #AF3240) at 1:400 dilution. Following treatment with biotinylated rabbit anti-goat secondary antibody at 1:400 dilution (Vector Laboratories, #BA-5000), the slides were developed using Vectastain Elite ABC (Vector Laboratories, #PK-6100) and diaminobenzidine (Dako Corporation, #K3468). Hemaoxylin (Richard-Allan Scientific, #2-7231) was used as a counterstain. Using a digital pathology system (ScanScope® CS and ImageScope™ software, Aperio), lesions in the epithelium (minimum ≧0.2 mm²) and areas in the underling stroma (minimum ≧0.2 mm²) were marked by a study pathologist. Representative normal regions were selected if no lesions remained. Cells with positive nuclear staining were counted using the software.

### HLA Typing

Low-resolution typing for HLA class I A, B, and C and class II DRB1, DQB1, and DPB1 was performed with MicroSSP Generic DNA Typing Trays (One Lambda, #SSP1L and #SSPDRQP1), using DNA extracted from PBMCs. Data were analyzed with HLA Fusion (One Lambda).

### Statistical Analysis

A generalized estimate equation analyses were performed to compare the frequencies of grade 2 immediate and delayed injection site reactions between the higher doses (250 and 500 ug) and the lower doses (50 and 100 ug), while accounting for the correlation among injections given to the same individual. A sign test was performed to compare the numbers of cervical quadrants with visible lesions prior to and after 4 vaccinations. A paired t-test was used to determine significance of increased CD3 T-cell responses as determined by rising positivity index for each region after 2 or 4 vaccinations, and to compare percentages of Th1, Th2, and Tregs after 2 or 4 vaccinations from the baseline. Wilcoxon rank-sum test was used to compare percentages of Th1, Th2, or Tregs between responders and non-responders prior to vaccination, after 2 vaccinations or after 4 vaccinations. Chi-square test was used to compare frequencies of each HLA molecule between the patients and the general population in the United States or between the patients and the corrected population frequencies based on racial distributions of the patients.⁴⁷ Fisher's exact text was used to compare HLA frequencies between responders and non-responders. No adjustments were made for multiple comparisons.

### REFERENCES FOR EXAMPLE 3

1. Cancer IAfRo. GLOBOCAN 2012 CANCER FACT SHEET. Cedex, France, 2012.
2. Chaturvedi A K. Beyond cervical cancer: burden of other HPV-related cancers among men and women. J Adolesc Health 2010; 46:S20-6.
3. Tota J E, Chevarie-Davis M, Richardson L A, Devries M, Franco E L. Epidemiology and burden of HPV infection and related diseases: implications for prevention strategies. Prev Med 2011; 53 Suppl 1:S12-21.
4. Bruinsma F J, Quinn M A. The risk of preterm birth following treatment for precancerous changes in the cervix: a systematic review and meta-analysis. BJOG 2011; 118:1031-41.
5. Massad L S, Einstein M H, Huh W K, Katki H A, Kinney W K, Schiffman M, Solomon D, Wentzensen N, Lawson H W. 2012 updated consensus guidelines for the management of abnormal cervical cancer screening tests and cancer precursors. Obstet Gynecol 2013; 121:829-46.
6. Kumar V, Fausto N, Abbas A. Cervix. Robbins & Cotran Pathologic Basis of Disease, 2004:1072-9.
7. Pirisi L, Yasumoto S, Feller M, Doniger J, DiPaolo J. Transformation of human fibroblasts and keratinocytes with human papillomavirus type 16 DNA. J Virol 1987; 61:1061-6.
8. Schlegel R, Phelps W C, Zhang Y L, Barbosa M. Quantitative keratinocyte assay detects two biological activities of human papillomavirus DNA and identifies viral types associated with cervical carcinoma. Embo J 1988; 7:3181-7.
9. Nakagawa M, Stites D P, Patel S, Farhat S, Scott M, Hills N K, Palefsky J M, Moscicki A B. Persistence of human papillomavirus type 16 infection is associated with lack of cytotoxic T lymphocyte response to the E6 antigens. J Infect Dis 2000; 182:595-8.
10. Kim K H, Greenfield W W, Cannon M J, Coleman H N, Spencer H J, Nakagawa M. CD4+ T-cell response against human papillomavirus type 16 E6 protein is associated with a favorable clinical trend. Cancer Immunol Immunother 2012; 61:63-70.
11. Nakagawa M, Gupta S K, Coleman H N, Sellers M A, Banken J A, Greenfield W W. A favorable clinical trend is associated with CD8 T-cell immune responses to the human papillomavirus type 16 e6 antigens in women being studied for abnormal pap smear results. J Low Genit Tract Dis 2010; 14:124-9.
12. Esch R E, Buckley C E, 3rd. A novel Candida albicans skin test antigen: efficacy and safety in man. J Biol Stand 1988; 16:33-43.
13. Clifton M M, Johnson S M, Roberson P K, Kincannon J, Horn T D. Immunotherapy for recalcitrant warts in children using intralesional mumps or Candida antigens. Pediatr Dermatol 2003; 20:268-71.
14. Horn T D, Johnson S M, Helm R M, Roberson PK. Intralesional immunotherapy of warts with mumps, Candida, and Trichophyton skin test antigens: a single-blinded, randomized, and controlled trial. Arch Dermatol 2005; 141:589-94.
15. Johnson S M, Roberson P K, Horn T D. Intralesional injection of mumps or Candida skin test antigens: a novel immunotherapy for warts. Arch Dermatol 2001; 137:451-5.
16. Kim K H, Horn T D, Pharis J, Kincannon J, Jones R, O'Bryan K, Myers J, Nakagawa M. Phase 1 clinical trial of intralesional injection of Candida antigen for the treatment of warts. Arch Dermatol 2010; 146:1431-3.
17. Wang X, Coleman H N, Nagarajan U, Spencer H J, Nakagawa M. Candida skin test reagent as a novel adjuvant for a human papillomavirus peptide-based therapeutic vaccine. Vaccine 2013; 31:5806-13.
18. Abbas A K, Lichtman A H, Pillai S. Cellular and Molecular Immunology. Philadelphia: Elsevier, 2011.

19. Nieminen P, Harper D M, Einstein M H, Garcia F, Donders G, Huh W, Wright T C, Stoler M, Ferenczy A, Rutman O, et al. Efficacy and safety of RO5217990 treatment in patients with high grade cervical intraepithelial neoplasia (CIN2/3). 28th International Papillomavirus Conference. Puerto Rico, 2012.

20. Kenter G G, Welters M J, Valentijn A R, Lowik M J, Berends-van der Meer D M, Vloon A P, Essahsah F, Fathers L M, Offringa R, Drijfhout J W, et al. Vaccination against HPV-16 oncoproteins for vulvar intraepithelial neoplasia. N Engl J Med 2009; 361:1838-47.
21. Daayana S, Elkord E, Winters U, Pawlita M, Roden R, Stern P L, Kitchener H C. Phase II trial of imiquimod and HPV therapeutic vaccination in patients with vulval intraepithelial neoplasia. Br J Cancer 2010; 102:1129-36.

22. Ribas A, Timmerman J M, Butterfield L H, Economou J S. Determinant spreading and tumor responses after peptide-based cancer immunotherapy. Trends Immunol 2003; 24:58-61.

23. Bruni L, Diaz M, Castellsague X, Ferrer E, Bosch F X, de Sanjose S. Cervical human papillomavirus prevalence in 5 continents: meta-analysis of 1 million women with normal cytological findings. J Infect Dis 2010; 202:1789-99.
24. Clifford G M, Gallus S, Herrero R, Munoz N, Snijders P J, Vaccarella S, Anh P T, Ferreccio C, Hieu N T, Matos E, et al. Worldwide distribution of human papillomavirus types in cytologically normal women in the International Agency for Research on Cancer HPV prevalence surveys: a pooled analysis. Lancet 2005; 366:991-8.
25. Clifford G M, Smith J S, Plummer M, Munoz N, Franceschi S. Human papillomavirus types in invasive cervical cancer worldwide: a meta-analysis. Br J Cancer 2003; 88:63-73.
26. Munoz N, Bosch F X, de Sanjose S, Herrero R, Castellsague X, Shah K V, Snijders P J, Meijer C J. Epidemiologic classification of human papillomavirus types associated with cervical cancer. N Engl J Med 2003; 348:518-27.
27. Smith J S, Lindsay L, Hoots B, Keys J, Franceschi S, Winer R, Clifford G M. Human papillomavirus type distribution in invasive cervical cancer and high-grade cervical lesions: a meta-analysis update. Int J Cancer 2007; 121:621-32.
28. van den Hende M, Redeker A, Kwappenberg K M, Franken K L, Drijfhout J W, Oostendorp J, Valentijn A R, Fathers L M, Welters M J, Melief C J, et al. Evaluation of immunological cross-reactivity between clade A9 high-risk human papillomavirus types on the basis of E6-Specific CD4+ memory T cell responses. J Infect Dis 2010; 202:1200-11.
29. Slingluff C L, Jr., Petroni G R, Olson W C, Smolkin M E, Ross M I, Haas N B, Grosh W W, Boisvert M E, Kirkwood J M, Chianese-Bullock K A. Effect of granulocyte/macrophage colony-stimulating factor on circulating CD8+ and CD4+ T-cell responses to a multipeptide melanoma vaccine: outcome of a multicenter randomized trial. Clin Cancer Res 2009; 15:7036-44.
30. Nakagawa M, Coleman H N, Wang X, Daniels J, Sikes J, Nagarajan U M. IL-12 secretion by Langerhans cells stimulated with Candida skin test reagent is mediated by dectin-1 in some healthy individuals. Cytokine 2014; 65:202-9.
31. Manetti R, Parronchi P, Giudizi M G, Piccinni M P, Maggi E, Trinchieri G, Romagnani S. Natural killer cell stimulatory factor (interleukin 12 [IL-12]) induces T helper type 1 (Th1)-specific immune responses and inhibits the development of IL-4-producing Th cells. J Exp Med 1993; 177:1199-204.
32. Hsieh C S, Macatonia S E, Tripp C S, Wolf S F, O'Garra A, Murphy K M. Development of TH1 CD4+ T cells through IL-12 produced by Listeria-induced macrophages. Science 1993; 260:547-9.
33. Tugues S, Burkhard S H, Ohs I, Vrohlings M, Nussbaum K, Vom Berg J, Kulig P, Becher B. New insights into IL-12-mediated tumor suppression. Cell Death Differ 2014.
34. Berd D, Maguire H C, Jr., Mastrangelo M J. Potentiation of human cell-mediated and humoral immunity by low-dose cyclophosphamide. Cancer Res 1984; 44:5439-43.
35. Emadi A, Jones R J, Brodsky R A. Cyclophosphamide and cancer: golden anniversary. Nat Rev Clin Oncol 2009; 6:638-47.
36. Hildesheim A, Schiffman M, Scott D R, Marti D, Kissner T, Sherman M E, Glass A G, Manos M M, Lorincz A T, Kurman R J, et al. Human leukocyte antigen class I/II alleles and development of human papillomavirus-related cervical neoplasia: results from a case-control study conducted in the United States. Cancer Epidemiol Biomarkers Prey 1998; 7:1035-41.
37. Madeleine M M, Johnson L G, Smith A G, Hansen J A, Nisperos B B, Li S, Zhao L P, Daling J R, Schwartz S M, Galloway D A. Comprehensive analysis of HLA-A, HLA-B, HLA-C, HLA-DRB1, and HLA-DQB1 loci and squamous cell cervical cancer risk. Cancer Res 2008; 68:3532-9.
38. Wang S S, Wheeler C M, Hildesheim A, Schiffman M, Herrero R, Bratti M C, Sherman M E, Alfaro M, Hutchinson M L, Morales J, et al. Human leukocyte antigen class I and II alleles and risk of cervical neoplasia: results from a population-based study in Costa Rica. J Infect Dis 2001; 184:1310-4.
39. Kim K H, Dishongh R, Santin A D, Cannon M J, Bellone S, Nakagawa M. Recognition of a cervical cancer derived tumor cell line by a human papillomavirus type 16 E6 52-61-specific CD8 T cell clone. Cancer Immun 2006; 6:9.
40. Wang X, Greenfield W W, Coleman H N, James L E, Nakagawa M. Use of Interferon-gamma Enzyme-linked Immunospot Assay to Characterize Novel T-cell Epitopes of Human Papillomavirus. J Vis Exp 2012.
41. Nakagawa M, Kim K H, Moscicki A B. Patterns of CD8 T-cell epitopes within the human papillomavirus type 16 (HPV 16) E6 protein among young women whose HPV 16 infection has become undetectable. Clin Diagn Lab Immunol 2005; 12:1003-5.
42. Nakagawa M, Kim K H, Gillam T M, Moscicki A B. HLA class I binding promiscuity of the CD8 T-cell epitopes of human papillomavirus type 16 E6 protein. J Virol 2007; 81:1412-23.
43. Wang X, Moscicki A B, Tsang L, Brockman A, Nakagawa M. Memory T cells specific for novel human papillomavirus type 16 (HPV16) E6 epitopes in women whose HPV16 infection has become undetectable. Clin Vaccine Immunol 2008; 15:937-45.
44. Kaul R, Dong T, Plummer F A, Kimani J, Rostron T, Kiama P, Njagi E, Irungu E, Farah B, Oyugi J, et al. CD8(+) lymphocytes respond to different HIV epitopes in seronegative and infected subjects. J Clin Invest 2001; 107:1303-10.
45. Kobayashi A, Weinberg V, Darragh T, Smith-McCune K. Evolving immunosuppressive microenvironment during human cervical carcinogenesis. Mucosal Immunol 2008; 1:412-20.
46. Magg T, Mannert J, Ellwart J W, Schmid I, Albert M H. Subcellular localization of FOXP3 in human regulatory and nonregulatory T cells. Eur J Immunol 2012; 42:1627-38.
47. OrganProcurementandTransplantationNetwork. U.S.Department of Health & Human Services.

### Example 4

A novel Prostate Cancer Immunotherapy Using Prostate Specific Antigen Peptides and Candida Skin Test Reagent as an Immunostimulant

### Abstract

Background Prostate cancer is the most common cancer and the second leading cause of cancer-related death among the male population in Western countries. Our group has shown that Candida skin test reagent can induce T-cell proliferation and interleukin-12 secretion by Langerhans cells in vitro. It has also been tested as a vaccine adjuvant for an investigational human papillomavirus therapeutic vaccine, and an increase in circulating T-helper type 1 (Th1) cells has been demonstrated in vaccine recipients. In the current work, the feasibility of using prostate specific antigen (PSA) peptides and Candida as a novel prostate cancer immunotherapy was evaluated. Methods Regions of PSA protein likely to dissolve in a single acidic solution were selected, and 85% of PSA amino acid sequences were synthesized in 6 peptides (amino acids 1-40, 41-80, 81-120, 161-200, 201-240, and 241-261). Solubility of these peptides in solutions compatible for human use, maturation effects of these peptides on Langerhans cells by fluorescent activated cell sorter analysis, and recognition of these peptides by peripheral immune cells from prostate cancer patients using interferon-gamma enzyme-linked immunospot (ELISPOT) assay were evaluated. Results The peptides were soluble in 10 mM succinate (pH5) with 5% glycine, and they demonstrated no maturation effects on Langerhans cells as determined by expression of CD40, CD80, CD86 and HLA-DR on the surface of monocyte-derived Langerhans cells from healthy donors. On the other hand, peripheral immune cells from 4 of 10 prostate cancer patients examined had positive responses in ELISPOT assay to one or more PSA peptide pools. Conclusions It would be feasible to use these PSA peptides and Candida as a novel prostate cancer immunotherapy, and a proportion of prostate cancer patients seem to have immune cells with the ability to recognize these PSA peptides already. Therefore, this immunotherapy may enhance immune responses to PSA potentially leading to tumor regression.

### Introduction

PSA is an ideal antigen for immunotherapy against prostate cancer as it is expressed in prostate but not in any other organs.

We prefer to use several peptides in the therapeutic vaccine, so a larger number of T-cell epitopes can be captured by the group. The peptides should be soluble together in a single solution. The peptides should contain a large number of T-cell epitopes, at least collectively. And they would preferably have the ability to mature Langerhans cells, since mature Langerhans cells promote T-cell activity.

We contemplated the PSA peptides of Table 5 for inclusion in a vaccine.

TABLE 5

| | Amino acid | |
|---|---|---|
| Peptide sequence | position | Charge |
| MWVPVVFLTL SVTWIGAAPL ILSRIVGGWE | 1-40 | 1 |
| CEKHSQPWQV (SEQ ID NO:2) | | |
| LVASRGRAVC GGVLVHPQWV LTAAHCIRNK | 41-80 | 8 |
| SVILLGRHSL (SEQ ID NO:3) | | |
| FHPEDTGQVF QVSHSFPHPL YDMSLLKNRF | 81-120 | 1 |
| LRPGDDSSHD (SEQ ID NO:4) | | |
| LMLLRLSEPA ELTDAVKVMD LPTQEPALGT | 121-160 | −3 |
| TCYASGWGSI (SEQ ID NO:5) | | |
| EPEEFLTPKK LQCVDLHVIS NDVCAQVHPQ | 161-200 | 1 |
| KVTKFMLCAG (SEQ ID NO:6) | | |
| RWTGGKSTCS GDSGGPLVCN GVLQGITSWG | 201-240 | 0 |
| SEPCALPERP (SEQ ID NO:7) | | |
| SLYTKVVHYR KWIKDTIVAN P (SEQ ID | 241-261 | 4 |
| NO: 8) | | |

The 121-160 peptide is the only one that is acidic (has a negative charge).

The hydrophobic/hydrophilic characteristics of the peptides are summarized in Table 6.

TABLE 6

| | Hydrophobic | Hydrophilic |
|---|---|---|
| Amino Acid | amino acid | amino acid |
| position | residues | residues |
| 1-40 | 12% | 60% |
| 41-80 | 20% | 50% |
| 81-120 | 32% | 38% |
| 121-160 | 18% | 50% |
| 161-200 | 28% | 45% |
| 201-240 | 15% | 32% |
| 241-261 | 29% | 43% |

The peptides corresponding to residues 1-40, 41-80, 81-120, 161-200, 201-240, and 241-261 were selected for inclusion in a therapeutic vaccine on the basis that they have neutral or positive charge and have less than 40% hydrophobic residues. Peptides 161-200 was not included because it has a negative charge.

### Peptide Solubility Testing

The six peptides 1-40, 41-80, 81-120, 161-200, 201-240, and 241-261 were tested individually for solubility at pH 4 or pH 5 with succinate (10 mM) or glutamate (10 mM). All except PSA 1-40 were soluble in both buffers. The turbidity of PSA 1-40 was lowest in 10 mM succinate pH 5 buffer. So solubility of PSA 1-40 was further tested in 10 mM succinate, pH 5 containing (1) 5% glycine, (2) 2% histidine, (3) 2.5% lysine, (4) 1.5% serine, (5) 1.5% threonine, or (6) 5% arginine. Of these buffers tested, PSA 1-40 was only soluble in 10 mM succinate, pH 5.0 with 5% glycine at 5.5 mg/ml.

In order to assess whether the 6 peptides combined can be solubilized in 10 mM succinate, pH5.0 with 5% glycine, attempts were made to solubilize each peptide at 20 mg/ml.

PSA 1-40 was insoluble at 20 mg/ml. Even after the peptide was then diluted to 5 mg/ml, it remained insoluble. However, the solubility was enhanced in the presence of other PSA peptides.

To solubilize all 6 peptides, PSA 1-40 was solubilized initially at 3 mg/ml and the other 5 peptides were solubilized at 7.5 mg/ml before all six solutions were combined so that the final concentration was 1 mg/ml/peptide in 10 mM succinate, 5% glycine, pH 5.0.

The solubility results as measured by turbidity at 630 nm of each of the 6 individual peptide solutions and the combined PSA 6 solution is shown in Table 7.

TABLE 7

| | | | PSA | PSA | PSA | PSA | | |
|---|---|---|---|---|---|---|---|---|
| Peptide | PSA | PSA | 81- | 161- | 201- | 241- | All 6 | |
| solution | 1-40 | 40-81 | 20 | 200 | 241 | 260 | peptides | Control |
| Turbidity | 0.695 | 0.041 | 0.037 | 0.039 | 0.153 | 0.143 | 0.077 | 0.037 |
| reading at | | | | | | | | |
| 630 nm | | | | | | | | |

### Assessing Maturation Effects of PSA Peptides

Purpose of the study: To examine whether Candin or peptides can stimulate the maturation of Langerhans cell. Surface expression of CD80, CD86, CD40 and HLA-DR were measured in Langerhans cells. CD40 and CD80 have been demonstrated to be critical for the activation of antigen-specific T-helper cells and cytotoxic T-cells.

### Methods

Peripheral blood mononuclear cells (PBMCs) were purified using the ficoll gradient centrifugation method from mononuclear cells collected from healthy donors (n=6) by apheresis.

Monocytes were then negatively isolated from PBMC using Monocyte Isolation Kit II and converted to Langerhans cells using granulocyte-macrophage colony-stimulating factor, IL-4, and transforming growth factor beta-1 over seven days. One million Langerhans cells each were then incubated with Candin, PSA 1-40, PSA 41-80, PSA 81-120, PSA 201-240, PSA 241-261, 5 peptides together, PSA 161-200, or 6 peptides together. Zymosan (10 mcg/ml, InvivoGen, San.Diego, Calif.), a yeast cell wall particle containing many polysaccha-rides including -glucan and mannan, was used as a positive control. Media containing no peptide served as a negative control. After 48 hours incubation, cells were stained with anti-human CD40 allophycocyanin, CD80 fluorescein isothiocyanate, CD86 PE-Cy5 and HLA-DR PE (eBioscience, San Diego, Calif.). Ten thousand events were acquired, and the data were analyzed usingFlowjo software (BD Biosciences).

### Results

### Langerhans Cell Maturation is not Promoted in Vitro by PSA Peptides

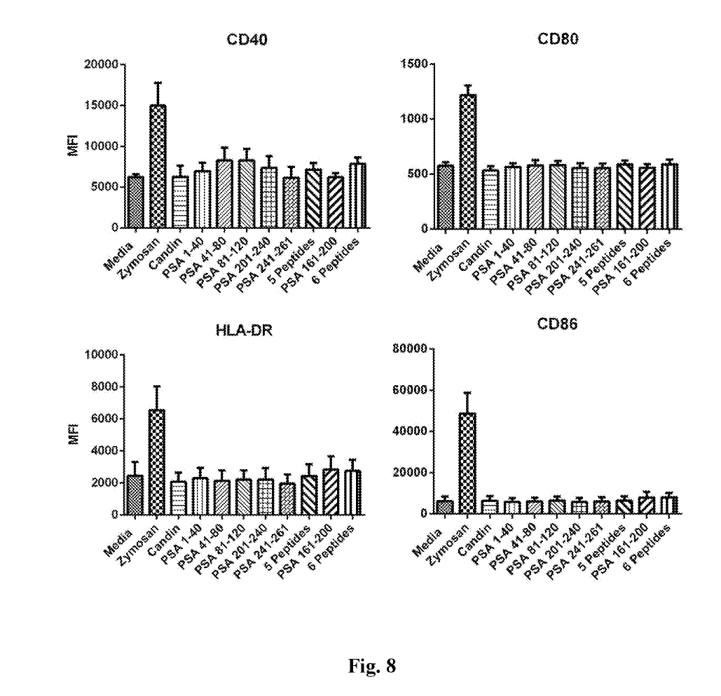

Maturation effects on Langerhans cells was examined by surface expression of CD40, CD80, CD86, and HLA-DR using antibodies as determined by mean fluorescence intensity in Langerhans cells exposed to PSA peptides, individually and combined at 48 hr. Dectin (Zymosan) was used as a positive control. An increase in surface expression of these markers indicates maturation of Langerhans cells. The fluorescent activated cell sorting (FACS) flow cytometry results are shown in FIG. 8. Neither CANDIN, nor any of the 6 tested peptides individually, nor the mixture of the 6 PSA peptides promoted Langerhans cell maturation as measured by CD40, CD80, CD86, or HLA-DR surface expression.
ELISPOT Assay for Immune Response to PSA Shows Immune Response to PSA in prostate Cancer Patients

A protocol of an enzyme-linked immunospot (ELISPOT) assay, which measures antigen-specific interferon-gamma secretion, previously used to monitor a clinical trial, was modified for this prostate cancer vaccine. Peripheral blood mononuclear cells (PBMCs) isolated from prostate cancer patients (n=10 patients) were analyzed. This assay is designed to answer the question of whether these peptides contain potential T-cell epitopes.

The PBMCs were incubated for 40 hours with one or more of the peptides, or with media alone as a negative control. The average spot-forming units (SFU) with each antigen were calculated. A response was considered positive when the average SFU in wells with a given peptide was at least twice that of the average SFU in the no-peptide control wells.

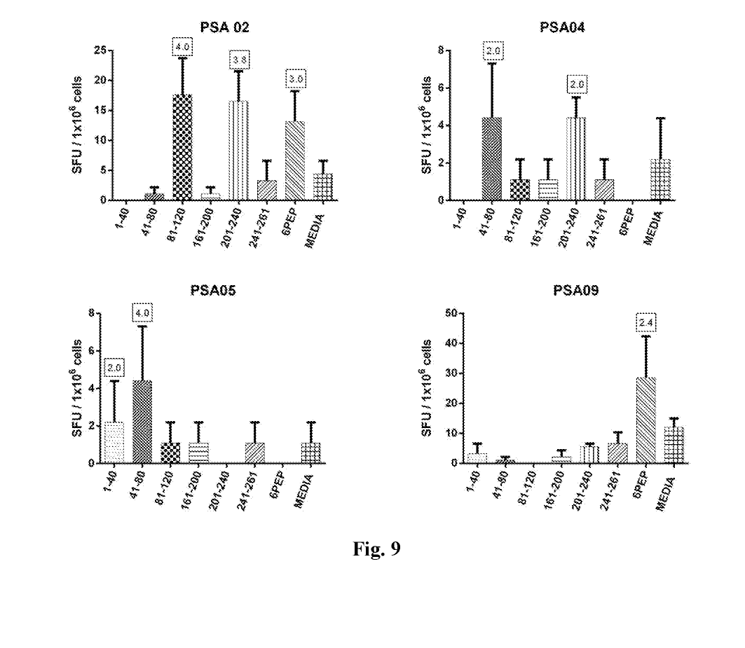

The results are shown in FIG. 9. Four of the patients had positive responses to at least one of the peptide antigens. PSA 41-80, PSA 201-240, and the 6-peptide mixture each produced a positive response in two patients.

### Discussion

The PSA peptides contacted with Langerhans cells in vitro did not induce maturation of the Langerhans cells. This is unlike the result with HPV E6 peptides described in Example 2 above.

We found that a portion (4 of 10) of prostate cancer patients have immune cells that recognize PSA antigens already (as shown by IFN-γ secretion), without having been vaccinated with PSA or any PSA peptides. This indicates that it is feasible to generate an immune response against the PSA peptides.

What is claimed is:

1. A composition comprising:
(a) a recall antigen, and
(b) one or more purified polypeptides or peptides comprising antigens specific for prostate tissue or prostate cancer; and
(c) a pharmaceutically acceptable diluent;
wherein the composition is sterile.

2. The composition of claim 1 wherein the component (b) is a purified polypeptide comprising prostate specific antigen (SEQ ID NO:1) or is one or more purified antigenic peptide fragments of SEQ ID NO:1.

3. The composition of claim 2 wherein the component (b) is one or more purified antigenic peptide fragments of SEQ ID NO:1, wherein the peptide fragments are 10-80 amino acid residues in length.

4. The composition of claim 3 wherein the one or more purified antigenic peptide fragments are selected from peptides consisting of residues 1-40, residues 41-80, 81-120, residues 161-200, residues 201-240, and residues 241-261 of SEQ ID NO:1.

5. The composition of claim 3 wherein component (b) is a plurality of antigenic peptide fragments of SEQ ID NO:1, wherein the peptide fragments are each 15-50 amino acid residues in length and collectively comprise residues 1-120 and 161-261 of SEQ ID NO:1.

6. The composition of claim 5 wherein the plurality of antigenic peptide fragments of SEQ ID NO:1 do not collectively comprise residues 121-160 of SEQ ID NO:1.

7. The composition of claim 5 wherein component (b) consists of peptide fragments of SEQ ID NO:1 consisting of residues 1-40, 41-80, 81-120, 201-240, and 241-261 of SEQ ID NO:1.

8. The composition of claim 1 wherein the peptides are acetylated on their N-terminus or amidated on their C-terminus, or both acetylated on their N-terminus and amidated on their C-terminus.

9. The composition of claim 1 wherein the recall antigen is candida extract, mumps antigen, or trichophyton extract.

10. The composition of claim 1 wherein the recall antigen is candida extract.

11. The composition of claim 3 wherein the peptide fragments collectively comprise more than 50% of the sequence of SEQ ID NO:1.

12. (canceled)

13. The composition of claim 1 wherein the composition has a pH of between 3.0 and 6.0.

14. The composition of claim 13 further comprising % (w/v) glycine.

15. A method of making the composition of claim 1 comprising filling vials with the composition in a sterile clean room.

16. A method of treating prostate cancer comprising:
intradermally injecting a person afflicted with prostate cancer with the composition of claim 1.

17. The method of claim 16 wherein the prostate cancer in the person is in partial or complete remission at the time of the intradermally injecting step, and the method is a method of preventing growth of the cancer or preventing recurrence of the cancer.

18. A method of preventing growth of prostate cancer tumors or preventing recurrence of prostate cancer in a mammal comprising:
injecting the composition of claim 1 intradermally in a mammal in need thereof;
wherein the mammal is afflicted with prostate cancer, or the mammal was afflicted with prostate cancer and the cancer is now in remission.

19. A composition comprising:
(a) a plurality of antigenic peptide fragments of SEQ ID NO:1, wherein the peptide fragments are each 15-50 amino acid residues in length and collectively comprise residues 1-120 and 161-261 of SEQ ID NO:1;
and
(b) a pharmaceutically acceptable diluent;
wherein the composition is sterile.

20. The composition of claim 19 wherein the plurality of antigenic peptide fragments of SEQ ID NO:1 do not collectively comprise residues 121-160 of SEQ ID NO:1.

21. (canceled)

22. The composition of claim 19 wherein the composition has a pH of between 3.0 and 6.0.

23. The composition of claim 22 further comprising at least 2% to 12% (w/v) glycine.

24. The composition of claim 19 further comprising a recall antigen.
